(19) United States
(12) Patent Application Publication
(10) Pub. No.: US 2023/0142800 A1
(43) Pub. Date: May 11, 2023

(54) Adalimumab Variants with Reduced Immunogenic Potential

(71) Applicant: COMMISSARIAT A L'ENERGIE ATOMIQUE ET AUX ENERGIES ALTERNATIVES, Paris (FR)

(72) Inventors: Hervé Nozach, Verrieres le Buisson (FR); Coline Sivelle, Risskov-Aarhus (DK); Raphaël Sierocki, Massy (FR); Bernard Maillere, Versailles (FR)

(73) Assignee: COMMISSARIAT A L'ENERGIE ATOMIQUE ET AUX ENERGIES ALTERNATIVES, Paris (FR)

(21) Appl. No.: 17/786,206

(22) PCT Filed: Dec. 17, 2020
(86) PCT No.: PCT/FR2020/052485

§ 371 Date: Dec. 5, 2022

(30) Foreign Application Priority Data
Dec. 17, 2019 (FR) FR19 14645

(51) Int. Cl. C07K 16/24; A61P 37/02

(52) U.S. Cl. CPC C07K 16/241; A61P 37/02; C07K 2317/565

(57) ABSTRACT

The invention relates to adalimumab variants with reduced immunogenic potential and retained or increased affinity and therapeutic applications thereof.

### FIELD OF THE INVENTION

The invention relates to adalimumab variants with reduced immunogenic potential and retained or increased affinity and therapeutic applications thereof.

### DESCRIPTION OF RELATED ART

Autoimmune inflammatory diseases affect a very large part of the population, especially in developed countries. Rheumatoid arthritis, which is one of the most widespread conditions, affects over 1 million people in the United States (Hunter, T. M. et al., Rheumatol. Int. (2017). doi:10.1007/s00296-017-3726-1). The anti-TNF alpha such as Humira® (adalimumab), a human IgG1 antibody neutralizing TNF alpha, constitute an effective symptomatic treatment for very many of these autoimmune inflammatory diseases. For this reason, adalimumab is today widely used in the treatment of ankylosing spondylitis, rheumatoid arthritis, hemorrhagic colitis, psoriatic arthritis, Crohn's disease, cutaneous psoriasis and juvenile arthritis (Feldmann, M. & Maini, Nat. Med. 9, 1245-1250 (2003)). Adalimumab has however been seen to be immunogenic in a non-negligible number of patients (up to 50% for some conditions) who produce antibodies directed against the therapeutic protein (ADA, for anti-drug antibody), detectable in the serum (Strand, V. et al., BioDrugs 31, 299-316 (2017)). The ADA result in the formation of immune complexes accelerating the elimination of the therapeutic antibodies. Clinically, the presence of ADA is inversely correlated with the response to the treatment. Patients having a good response to the treatment are those in whom the tests do not reveal any ADA (van Schouwenburg, P. A., Rispens, T. & Wolbink, G. J., Nat. Rev. Rheumatol. 9, 164-172 (2013)).

Clinically, the ADA problem is treated by different approaches. It was observed that the effect of the ADA was reduced by increasing the doses administered. Thus the treatments are often done with doses that increase in step with the patients' immunity. On the other hand, the therapies are often co-administered with methotrexate, since this immunosuppressant inhibits the ADA production and distinctly improved results can be obtained. These measures are however unsatisfactory in so far as the patients continue just the same to get immunity against the protein (van Schouwenburg, P. A., Rispens, T. & Wolbink, G. J., Nat. Rev. Allergy Immunol. 38, 82-89 (2010); Radstake, T. R. D. J., Ann. Rheum. Dis., 68, 1739-1745 (2009)).

The suppression of T cell epitopes by disruption of the interaction with the HLA II molecules, called de-immunization, was shown to be an effective method for reducing the immunogenicity of proteins with therapeutic purpose, such as enzymes and immunotoxins (Mazor, R. et al. Oncotarget 7, 29916-29926 (2016); Cantor, J. R. et al., Proc. Natl. Acad. Sci. 108, 1272-1277 (2011); Ettinger, R. A. et al., Blood Adv. 2, 309-322 (2018); Mazor, R. et al., Proc. Natl. Acad. Sci. U.S.A. 109, E3597-603 (2012)).

Different T cell epitopes were previously identified in the adalimumab sequence (Meunier, S. et al., Cellular & Molecular Immunology, 2019, Oct. 28. doi: 10.1038/s41423-019-0304-3). They are mainly located on the heavy chain of the antibody on which they are distributed in two regions. The majority of the T cell epitopes are concentrated in the first region and overlap extensively with CDR-H3 (L82C to T107 residues using Kabat numbering; FIG. 1). The second region is located near CDR-H2 and comprises two T cell epitopes (residues D46 to E64 using Kabat numbering; FIG. 1). These T cell epitopes are potentially the origin of the adalimumab immunogenicity. Since these have significant repercussions for patients, the development of alternatives less immunogenic than adalimumab seems to be a necessity.

However, this approach of de-immunization by elimination of T cell epitopes is not suited to therapeutic antibodies which are humanized or human antibodies, since T cell epitopes thereof are mainly present near the CDR which are essential for the biological activity of the therapeutic antibody (Harding et al., mAbs 2, 256-265 (2010)). In fact, the CDR are what determine the specificity and also the affinity of the antibody for the target antigen of the therapeutic antibody.

Adalimumab variants in which the framework regions (FR) from the variable domains of the heavy and light chains were replaced by less immunogenic framework regions from other human immunoglobulins G (IgG) are described in application EP 3,178,487. Such variants do not have a satisfactory de-immunization for therapeutic use since they comprise the majority of the T cell epitopes present in the CDR.

Adalimumab variants comprising mutations in a region containing suspected CD4 T cell epitopes extending on both sides of the CDR1 of the light chains (CDR-L1; positions C23 to K45, using Kabat numbering) are described in the application WO 2010/121140. Despite the choice of mutations for not significantly reducing the affinity of the variants, all the resulting variants had a reduced affinity for TNF alpha compared to adalimumab, where this reduction of affinity was drastic (at least 50%) for most (70%) of the resulting variants.

Consequently, there is a need to have new adalimumab variants which are better suited to their therapeutic use in that they have both a reduced immunogenic potential and an intact affinity.

### BRIEF SUMMARY OF THE INVENTION

The inventors have identified mutations in the immunogenic regions overlapping the CDR-H2 (or CDRH2) and CDR-H3 (or CDRH3) regions of adalimumab which reduce the immunogenicity while maintaining the TNF alpha binding affinity. They isolated variants from combinatorial libraries which surprisingly have a reduced immunogenic potential and a TNF alpha binding affinity greater than that of adalimumab. Some of the variants have an affinity 5 to 50 times greater than that of adalimumab. To the extent where the biological activity of the anti-TNF antibody—specifically the neutralization of the TNF alpha—depends on the affinity thereof for TNF, it can be expected that the variants from the invention will have a biological activity greater than that of adalimumab.

Consequently, the object of the present invention is a variant of a therapeutic anti-TNF alpha antibody comprising variable domains VH and VL of sequences SEQ ID NO: 1 and SEQ ID NO: 2, said variant comprising at least two amino acid substitutions in at least one sequence overlapping one of the CDRH2 or CDRH3 regions determining the complementarity of said VH variable domain; where said at least two amino acid substitutions in the sequence overlapping the CDRH2 region are selected from the group consisting of:
the substitution of S49 by another amino acid selected from A or G;
preferably G;
the substitution of A50 by another amino acid selected from G, S, T or D;
the substitution of T52 by another amino acid selected from A, N or S;
preferably N or S;
the substitution S54G;
the substitution of I57 by another amino acid selected from A, H, N, Q, R, S, T or W; preferably A, H, S, N, Q, R or T, preferably R, H, S or T; and when the variant comprises the residue A49 then it also comprises the residue N52, S52 or T52 and the residue G54;
where said at least two amino acid substitutions in the sequence overlapping the CDRH3 region are selected from the group consisting of:
the substitution of V89 by L;
the substitution of V95 by another amino acid selected from A, S or T;
the substitution of S96 by another amino acid selected from A, G, H, K, N, Q, R or T; preferably T, Q, N or H;
the substitution of Y97 by H;
the substitution of L98 by T;
the substitution of S99 by P;
the substitution of T100 by another amino acid selected from P or 5,
   with the exclusion of variants comprising the residues V89 or L89, V95, K96, Y97, L98, P99 and S100, V89, V95, A96, Y97, L98, P99 and S100; the positions of said amino acid residues being indicated with reference to the Kabat numbering; and said variant presenting a reduced immunogenic potential and a binding affinity for TNF alpha at least equal to or superior compared to the therapeutic anti-TNF alpha antibody from which it is derived.

According to an embodiment of the invention, said variant comprises at least three substitutions in one of the sequences overlapping the CDRH2 or CDRH3 region; preferably in each of said sequences overlapping the CDRH2 and CDRH3 regions.

According to an embodiment of the invention, said variant comprises a combination of substitutions in the sequence overlapping the CDRH2 region selected from:
S54G and I57R;
T52N or T52S, S54G and I57T, I57R, I57Q or I57H;
S49G, T52N and I57H;
S49A or S49G, S54G and I57T or I57R;
S49G, T52N, T52S or T52A; S54G; and I57T, I57R, I57H, I57S, I57Q or I57N; and possibly A50T, A50G or A50S;
S49A, T52N or T52S; S54G; and I57T, I57R, I57H, I57Q, I57S or I57A; and
S49G, A50G, S54G and I57R.

According to a preferred embodiment of the invention, said variant comprises a combination of substitutions in the sequence overlapping the CDRH2 region selected from:
(i) S49G, T52N and I57H; S49A, S54G and I57T; S49G, S54G and I57R; T52N, S54G and I57T;
(ii) S49G, T52N, S54G and I57R; S49G, T52N, S54G and I57H; S49G, T52N, S54G and I57T; S49G, T52N, S54G and I57S; S49G, A50G, T52N and I57H; S49G, T52S, S54G and I57R; S49A, T52N, S54G and I57T; S49G, T52S, S54G and I57N; S49G, T52S, S54G and I57Q; S49G, A50G, S54G and I57R, S49G, T52S, S54G and I57H; S49G, T52S, S54G and I57T; S49G, T52S, S54G and I57S; S49A, T52N, S54G and I57H; and
(iii) S49G, A50T, T52N, S54G and I57S; S49G, A50G, T52N, S54G and I57R; S49G, A50S, T52N, S54G and I57R; S49G, A50D, T52S, S54G and I57T; S49G, A50G, T52S, S54G and I57R; S49G, A50S, T52A, S54G and I57H; S49G, A50S, T52S, S54G and I57R; S49G, A50S, T52A, S54G and I57T; and S49G, A50S, T52A, S54G and I57S;
   and preferably selected from: S49G, T52N, S54G and I57R; S49G, A50T, T52N, S54G and I57S; S49G, T52N, S54G and I57H; S49G, T52N and I57H; S49G, A50D, T52S, S54G and I57T.

According to a preferred embodiment of the invention, said variant comprises a combination of substitutions in the sequence overlapping the CDRH3 region selected from:
V95S, V95T or V95A; and
S96T, S96Q, S96N or S96H; and
S99P; and possibly V89L.

According to a preferred embodiment of the invention, said variant comprises a combination of substitutions in the sequence overlapping the CDRH3 region selected from:
(a) S96K and S99P;
(b) V95T, S96T and S99P; V95T, S96K and S99P; V95T, S96R and S99P; V95T, S99P and T100S, V89L, S96K and S99P; S96T, S99P and T100S, S96K, S99P; V95A, S96K and S99P; V95S, S96K and S99P; V95T, S96G and S99P; S96K, S99P and T100P, V95T, S96H and S99P; V95T, S96N and S99P; V95S, S96Q and S99P; V95A, S96H and S99P; (c) V89L, V95T, S96N and S99P; V95T, S96T, S99P and T100S, V95A, S96H, Y97H and S99P; V89L, S96K, L98T, S99P; S96T, L98T, S99P and T100S, V89L, V95T, S96K and S99P; V95T, S96K, S99P and T100S, V95T, S96R L98T and S99P; V89L, V95T, S96R and S99P; V89L; V95T, S96T and S99P;
(d) V89L, V95T, S96T, S99P and T100S, V89L, S96K, L98T and S99P; V89L, V95T, S96K, S99P and T100S, V89L, V95T, S96R, S99P and T100S.

According to a more preferred embodiment of the invention, said variant comprises a combination of substitutions in the sequence overlapping the CDRH3 region selected from: V95T, S96T and S99P; V95T, S96N and S99P; V95S, S96Q and S99P; V95A, S96H and S99P; V95T, S96G and S99P; S96T, L98T, S99P and T100S, V95T, S96R, L98T and S99P; S96K, S99P and T100P, V89L, V95T, S96T and S99P; preferably selected from: V95T, S96T and S99P; V95T, S96N and S99P; V95S, S96Q and S99P; V95A, S96H and S99P; V89L, V95T, S96T and S99P.

According to a preferred embodiment of the invention, said variant comprises one of the following combinations of substitutions in the sequences overlapping the CDRH2 and CDRH3 regions:
S49G, T52N, S54G, I57R, V95S, S96Q and S99P;
S49G, A50T, T52N, S54G, I57S, V95T, S96T and S99P;
S49G, T52N, S54G, I57H, V95T, S96T and S99P;
S49G, T52N and I57H, V95T, S96T and S99P;
S49G, A50D, T52S, S54G and I57T, V95T, S96T and S99P;
S49G, T52N, S54G, I57R, V89L, V95T, S96T and S99P;
S49G, T52N, S54G, I57R, V95T, S96N and S99P;
S49G, T52N, S54G, I57R, V95A, S96H and S99P.

According to an embodiment of the invention, said variant further comprises the substitution R90K in the region CDRL3 determining the complementarity of the variable domain VL.

According to an embodiment of the invention, said variant comprises a human IgG heavy chain and a human Kappa light chain.

According to an embodiment of the invention, said variant is derived from adalimumab.

According to a preferred embodiment of the invention, said variant comprises a light chain of sequence SEQ ID NO: 2 or 32 and a heavy chain of sequence SEQ ID NO: 24 to 31.

Another aspect of the invention relates to an expression vector comprising a polynucleotide coding for a variant according to the invention.

Another aspect of the invention relates to a pharmaceutical composition comprising at least one variant according to the invention or a vector according to the invention, and a pharmaceutically acceptable vehicle and/or a carrier substance.

Another aspect of the invention relates to a composition according to the invention for use in the treatment of an inflammatory or autoimmune disease.

### DISCLOSURE OF THE INVENTION

The object of the present invention is an anti-TNF alpha therapeutic antibody comprising variable domains VH and VL of sequences SEQ ID NO: 1 and SEQ ID NO: 2, where the variant comprises a reduced immunogenic potential and a TNF alpha binding affinity at least equal to or superior compared to the therapeutic anti-TNF alpha antibody from which it is derived.

Because of the binding affinity that is at least equal or superior, it can be expected that the variant according to the invention has a biological activity at least equal or superior than that of the parent antibody.

The sequence SEQ ID NO: 1 correspondence to the heavy-chain variable domain (VH) of adalimumab and the sequence SEQ ID NO: 2 the sequence of the light-chain variable domain (VL) of adalimumab (FIG. 1). The sequence of the adalimumab heavy chain corresponds to the sequence SEQ ID NO: 3 and the sequence of the adalimumab light chain corresponds to the sequence SEQ ID NO: 4.

### Definitions

Therapeutic antibody is understood to mean a human or humanized antibody.

Antibody is understood to mean a whole antibody, an antibody fragment containing at least one antigen-binding domain or a molecule derived from an antibody.

TNF alpha or TNFalpha (“Tumor Necrosis Factor Alpha”) is understood to mean a multifunctional pro-inflammatory cytokine predominantly produced by monocytes and macrophages. Preferably, it means a human TNF alpha. Human TNF alpha corresponds to the GenBank QCI55793.1 sequence.

The terms “variant” and “mutant” are used interchangeably.

As it relates to an antibody variant according to the invention, reduced immunogenic potential is understood to mean a reduction of the number of HLA II molecules which can be bound by at least one of the CD4 T cell epitopes of the variant as compared to the parent antibody from which it is derived. The number of HLA II molecules which can be bound by a variant according to the invention is evaluated according to the standard techniques known to the person skilled in the art such as those described in particular in the examples. In particular it involves in silicone methods using CMH-II binding prediction tools such as the netMHCllpan 3.2 algorithm. (Jensen, K. K. et al., Immunology 154, 394-406 (2018)). HLA II binding is expressed in the form of the score defined by:

$$HLA II binding score = {\sum }_{j = 1}^{n} {\sum }_{i = 1}^{m} {Core}_{i , j} \qquad [ Math 1 ]$$

where the core is a nonhuman binding core predicted by netMHCllpan3.2; core_{>20%}=0 and core_{<20%}=1. i is the anchoring position of the core and j is the allele for which the core is predicted. The variant according to the invention is characterized by an HLA II binding score reduced by at least 10% (1.1 times or factor of 1.1) relative to the parent antibody from which it is derived.

The TNF alpha binding affinity of the variant is evaluated according to the standard techniques known to the person skilled in the art such as those described in particular in the examples. The affinity may be evaluated by the value of the equilibrium dissociation constant K_{D} of the variant, measured by conventional techniques such as described in the examples. The affinity may also be evaluated by the relative enrichment factor of the variant relative to the parent antibody which corresponds to the ratio between the enrichment values of the variant and the parent antibody such as defined in the examples. The variant according to the invention is characterized by a relative enrichment factor greater than or equal to 5 or a K_{D} at least 1.1 times less (less by a factor of 1.1 or by 10%) compared to the parent antibody from which it is derived.

An individual is understood to mean a human or animal individual, preferably a human individual.

Amino acids are indicated with the letter code.

The positions of the amino acid residues are indicated with reference to the Kabat numbering (FIG. 1).
Unless otherwise indicated, one, means at least one, and or means and/or.

According to an embodiment of the invention, said variant comprises at least two amino acid substitutions in at least one sequence overlapping the CDRH2 or CDRH3 regions determining the complementarity of said variable domain VH. The sequence overlapping the CDRH2 region extends from the residues E46 to C64 according to the Kabat numbering (SEQ ID NO: 8). The sequence overlapping the CDRH3 region extends from the residues V89 to G104 according to the Kabat numbering (SEQ ID NO: 9). Said variant advantageously comprises at least two substitutions in each of the two sequences overlapping the CDRH2 or CDRH3 region. Preferably, said variant comprises at least three substitutions, generally 3, 4 or 5 substitutions in one of the overlapping sequences; preferably, in each of the overlapping sequences of the CDRH2 and CDRH3 regions.

According to a preferred embodiment of the invention said at least two amino acid substitutions in the sequence overlapping the CDRH2 region are selected from the group consisting of:
the substitution of S49 by another amino acid selected from, A or G;
the substitution of A50 by another amino acid selected from G, S, T or D,
the substitution of T52 by another amino acid selected from, A, N or S;
the substitution S54G;
the substitution of I57 by another amino acid selected from A, H, N, Q, R, S, T, or W; and when the variant comprises the residue A49 then it also comprises the residue N52, S52 or T52 and the residue G54; and wherein the positions of said amino acid residues are indicated with referencing to the Kabat numbering.

The variants according to the invention are functional variants, meaning that they comprise a reduced immunogenic potential and a TNF alpha binding affinity at least equal or better, compared to the therapeutic anti-TNF alpha antibody from which it is derived. The invention excludes nonfunctional variants such as in particular variants comprising only two substitutions selected from T52N and A50G or T52N and I57T.

Preferably,
S49 is substituted by G;
T52 is substituted by N or S; and
I57 is substituted by A, H, S, N, Q, R, T, preferably R, H, S or T.

Advantageously, said substitutions in the sequence overlapping the CDRH2 region are selected from substitutions in positions S49, A50, T52, S54, H56 and I57. Preferably, said substitutions are in positions S54 and I57; S49, T52 and I57; S49, S54 and I57; T52, S54 and I57; S49, A50, T52 and I57; S49, A50, S54 and I57; S49, T52, S54 and I57; A50, T52, S54 and I57; or S49, A50, T52, S54 and I57; preferably in positions S49, T52 and I57; S49, S54 and I57; T52, S54 and I57; S49, A50, S54 and I57; S49, T52, S54 and I57; A50, T52, S54 and I57; or S49, A50, T52, S54 and I57.

Preferably, said variant comprises a combination of substitutions in the sequence overlapping the CDRH2 region selected from:
S54G and I57R;
T52N or T52S, S54G and I57T, I57R, I57Q or I57H;
S49G, T52N and I57H;
S49A or S49G, S54G and I57T or I57R;
S49G, T52N, T52S or T52A; S54G; and I57T, I57R, I57H, I57S, I57Q or I57N; and possibly A50T, A50G or A50S;
S49A, T52N or T52S; S54G; and I57T, I57R, I57H, I57Q, I57S or I57A;
S49G, A50G, S54G and I57R.

Preferably, said variant comprises a combination of substitutions in the sequence overlapping the CDRH2 region selected from:
(a) S54G and I57R;
(b) S49G, T52N and I57H;
(c) S49A, S54G and I57T; S49A, S54G and I57R; S49G, S54G and I57R
(d) T52S, S54G and I57R; T52S, S54G and I57Q; T52S, S54G and I57T; T52S; S54G and I57H; T52N, S54G and I57R; T52N, S54G and I57T;
(e) S49G, A50G, T52N and I57H;
(f) S49G, A50G, S54G and I57R;
(g) S49A, T52N, S54G and I57R; S49A, T52N, S54G and I57T; S49A, T52N, S54G and I57H; S49A, T52S, S54G and I57R; S49G, T52S, S54G and I57R; S49G, T52N, S54G and I57R; S49A, T52S, S54G and I57T; S49A, T52S, S54G and I57Q; S49A, T52S, S54G and I57H; S49G, T52S, S54G and I57T; S49A, T52N, S54G and I57H; S49A, T52S, S54G and I57S; S49A, T52S, S54G and I57A; S49G, T52S, S54G and I57H; S49G, T52S, S54G and I57R; S49G, T52S, S54G and I57N; S49G, T52S, S54G and I57Q; S49G, T52S, S54G and I57T; S49G, T52S, S54G and I57S; S49G, T52N, S54G and I57T; S49G, T52N, S54G and I57S; S49G, T52N, S54G and I57H;
(h) A50S, T52A, S54G and I57W; and
(i) S49G, A50T, T52N, S54G and I57S; S49G, A50G, T52N, S54G and I57R; S49G, A50S, T52N, S54G and I57R; S49G, A50D, T52S, S54G and I57T; S49G, A50G, T52S, S54G and I57R; S49G, A50S, T52A, S54G and I57H; S49G, A50S, T52S, S54G and I57R; S49G, A50S, T52A, S54G and I57T; and S49G, A50S, T52A, S54G and I57S.

Even more preferably, said variant comprises a combination of substitutions in the sequence overlapping the CDRH2 region selected from:
(i) S49G, T52N and I57H; S49A, S54G and I57T; S49G, S54G and I57R; T52N, S54G and I57T;
(ii) S49G, T52N, S54G and I57R; S49G, T52N, S54G and I57H; S49G, T52N, S54G and I57T; S49G, T52N, S54G and I57S; S49G, A50G, T52N and I57H; S49G, T52S, S54G and I57R; S49A, T52N, S54G and I57T; S49G, T52S, S54G and I57N; S49G, T52S, S54G and I57Q; S49G, A50G, S54G and I57R, S49G, T52S, S54G and I57H; S49G, T52S, S54G and I57T; S49G, T52S, S54G and I57S; S49A, T52N, S54G and I57H; and (iii) S49G, A50T, T52N, S54G and I57S; S49G, A50G, T52N, S54G and I57R; S49G, A50S, T52N, S54G and I57R; S49G, A50D, T52S, S54G and I57T; S49G, A50G, T52S, S54G and I57R; S49G, A50S, T52A, S54G and I57H; S49G, A50S, T52S, S54G and I57R; S49G, A50S, T52A, S54G and I57T; and S49G, A50S, T52A, S54G and I57S.

Particularly preferred variants according to the invention comprise one of the following combinations of substitutions in the sequence overlapping the CDRH2 region: S49G, T52N, S54G and I57R; S49G, A50T, T52N, S54G and I57S; S49G, T52N, S54G and I57H; S49G, T52N and I57H; S49G, A50D, T52S, S54G and I57T.

According to a preferred embodiment of the invention said at least two amino acid substitutions in the sequence overlapping the CDRH3 region are selected from the group consisting of:
the substitution of V89 by L;
the substitution of V95 by another amino acid selected from A, S or T;
the substitution of S96 by another amino acid selected from A, G, H, K, N, Q, R or T,
the substitution of Y97 by H;
the substitution of L98 by T;
the substitution of S99 by P,
the substitution of T100 by another amino acid selected from P or S;
   excluding variants comprising the residues V89 or L89, V95, K96, Y97, L98, P99 and S100, V89, V95, A96, Y97, L98, P99 and S100; wherein the positions of said amino acid residues are indicated with reference to the Kabat numbering.

Preferably, S96 is substituted by T, Q, N or H.

Advantageously, said substitutions in the sequence overlapping the CDRH3 region are selected from substitutions in positions: V89, V95, S96, Y97, L98, S99 and T100. Preferably, said substitutions are in the positions V89, V95, S96 and S99 or V95, S96 and S99.

Preferably, said variant comprises a combination of substitutions in the sequence overlapping the CDRH3 region selected from:
V95S, V95T or V95A; and
S96T, S96Q, S96N or S96H; and
S99P; and possibly V89L.

Advantageously, said variant comprises a combination of substitutions in the sequence overlapping the CDRH3 region selected from:
(a) S96K and S99P;
(b) V95T, S96T and S99P; V95T, S96K and S99P; V95T, S96R and S99P; V95T, S99P and T100S, V89L, S96K and S99P; S96T, S99P and T100S, S96K, S99P; V95A, S96K and S99P; V95S, S96K and S99P; V95T, S96G and S99P; S96K, S99P and T100P, V95T, S96H and S99P; V95T, S96N and S99P; V95S, S96Q and S99P; V95A, S96H and S99P;
(c) V89L, V95T, S96N and S99P; V95T, S96T, S99P and T100S, V95A, S96H, Y97H and S99P; V89L, S96K, L98T, S99P; S96T, L98T, S99P and T100S, V89L, V95T, S96K and S99P; V95T, S96K, S99P and T100S, V95T, S96R L98T and S99P; V89L, V95T, S96R and S99P; V89L; V95T, S96T and S99P;
(d) V89L, V95T, S96T, S99P and T100S; V89L, S96K, L98T and S99P; V89L, V95T, S96K, S99P and T100S; V89L, V95T, S96R, S99P and T100S.

More preferably, said variant comprises a combination of substitutions in the sequence overlapping the CDRH3 region selected from: V95T, S96T and S99P; V95T, S96N and S99P; V95S, S96Q and S99P; V95A, S96H and S99P; V95T, S96G and S99P; S96T, L98T, S99P and T100S; V95T, S96R, L98T and S99P; S96K, S99P and T100P; V89L, V95T, S96T and S99P.

Particularly preferred variants according to the invention comprise one of the following combinations of substitutions in the sequence overlapping the CDRH3 region: V95T, S96T and S99P; V95T, S96N and S99P; V95S, S96Q and S99P; V95A, S96H and S99P; V89L, V95T, S96T and S99P.

According to a preferred embodiment of the invention, said variant comprises at least two substitutions in each of the two sequences overlapping the CDRH2 or CDRH3 region such as defined above. Preferably said variant comprises a combination of substitutions in the sequence overlapping the CDRH2 region and a combination of substitutions in the sequence overlapping the CDRH3 region selected from the combinations such as defined above.

Particularly preferred embodiments according to the invention comprise:
one of the following combinations of substitutions in the sequence overlapping the CDRH2 region: S49G, T52N, S54G and I57R; S49G, A50T, T52N, S54G and I57S; S49G, T52N, S54G and I57H; S49G, T52N and I57H; S49G, A50D, T52S, S54G and I57T; and
one of the following combinations of substitutions in the sequence overlapping the CDRH3 region: V95T, S96T and S99P; V95T, S96N and S99P; V95S, S96Q and S99P; V95A, S96H and S99P; V89L, V95T, S96T and S99P.

Examples of particularly preferred variants according to the invention comprise one of the following combinations of substitutions in the sequences overlapping the CDRH2 and CDRH3 region:
S49G, T52N, S54G, I57R, V95S, S96Q and S99P;
S49G, A50T, T52N, S54G, I57S, V95T, S96T and S99P;
S49G, T52N, S54G, I57H, V95T, S96T and S99P;
S49G, T52N and I57H, V95T, S96T and S99P;
S49G, A50D, T52S, S54G and I57T, V95T, S96T and S99P;
S49G, T52N, S54G, I57R, V89L, V95T, S96T and S99P;
S49G, T52N, S54G, I57R, V95T, S96N and S99P;
S49G, T52N, S54G, I57R, V95A, S96H and S99P.

According to an embodiment of the invention, said variant further comprises the substitution R90K in the region CDRL3 determining the complementarity of the variable domain VL.

The variant according to the invention comprises a human immunoglobulin heavy chain of any isotype or class, preferably an IgG, preferably an IgG1. The variant according to the invention also comprises a human immunoglobulin light chain of any class, preferably a human Kappa light chain.

According to a preferred embodiment of the invention, said variant is derived from adalimumab. Preferably, said variant is selected from the group consisting of:
an adalimumab variant comprising a light chain of sequence SEQ ID NO: 2 or 32 and a heavy chain of sequence SEQ ID NO: 24 corresponding to mutation 1 from the examples;
an adalimumab variant comprising a light chain of SEQ ID NO: 2 or 32 and a heavy chain of sequence SEQ ID NO: 25 corresponding to mutation 2 from the examples;
an adalimumab variant comprising a light chain of sequence SEQ ID NO: 2 or 32 and a heavy chain of sequence SEQ ID NO: 26 corresponding to mutation 3 from the examples;
an adalimumab variant comprising a light chain of sequence SEQ ID NO: 2 or 32 and a heavy chain of sequence SEQ ID NO: 27 corresponding to mutation 4 from the examples;
an adalimumab variant comprising a light chain of sequence SEQ ID NO: 2 or 32 and a heavy chain of sequence SEQ ID NO: 28 corresponding to mutation 5 from the examples;
an adalimumab variant comprising a light chain of sequence SEQ ID NO: 2 or 32 and a heavy chain of sequence SEQ ID NO: 29 corresponding to mutation 6 from the examples;
an adalimumab variant comprising a light chain of sequence SEQ ID NO: 2 or 32 and a heavy chain of sequence SEQ ID NO: 30 corresponding to mutation 7 from the examples; and
an adalimumab variant comprising a light chain of sequence SEQ ID NO: 2 or 32 and a heavy chain of sequence SEQ ID NO: 31 corresponding to mutation 8 from the examples.

According to a preferred embodiment of the invention, said variant is characterized by an HLA II binding score reduced at least 1.5; 2; 2.5; 3; 3.5; 4; 4.5; 5 times or more compared to the parent antibody from which it is derived.

According to a preferred embodiment of the invention, said variant is characterized by a relative enrichment factor of at least 10¹ á 10⁷ (10¹, 5·10¹, 10², 5·10², 10³, 5·10³, 10⁴, 5·10⁴, 10⁵, 5·10⁵, 10⁶, 5·10⁶, 10⁷) or a K_{D} at least 1.2 to 40 times lower (2, 5, 10, 15, 20, 25, 30, 35) compared to the parent antibody from which it is derived.

The present invention also encompasses variants further comprising, at least one mutation (insertion, deletion, substitution) of additional amino acid and/or at least one function-conserving modification, meaning which preserves the reduced immunogenic potential and the greater or equal affinity of the variant. The following can be listed in particular among the additional mutations:
the substitution of A60 by another amino acid selected from S, N or D,
the substitution of D61 by another amino acid selected from P, G, T, N, Q, E, H or K; and
the substitution of S62 by another amino acid selected from F, Y, W, A, G, Q, D or E;
the substitution of A100A by 5,
the substitution of S100B by G, N or D,
the substitution of S1000 by Y, W, P or Q; and
the substitution of Y102 par W, I, V, A, G, S, T, Q, E, H, K or IR,
the substitution of T52 by G;
the substitution of I57 by another amino acid selected from E, K, Y or V;
the substitution of V89 by T;
the substitution of V95 by another amino acid selected from G, N or P, —the substitution of Y97 by W;
the substitution of L98 by another amino acid selected from F, Y, M or H;

and;
the substitution of S99 by T; and combinations of the preceding substitutions.

The variant may be modified by the introduction of any function-conserving modification in the area of the amino acid residue(s), peptide bond or end peptides. This or these modification(s), in particular one or more chemical modifications are made in the peptides by conventional methods known to the person skilled in the art, in particular including: merging the sequence of the variant with that of a polypeptide (label useful for purification of the variant, in particular in form's political by a protease) or a protein of interest, and the coupling of a molecule or an agent of interest. For example, the antibody may be coupled to a PEG molecule by the conventional methods known to the person skilled in the art.

The variant is in the form of a whole antibody, an antibody fragment containing at least one antigen-binding domain or a molecule derived from an antibody. The whole antibodies may be of any isotype, in particular human isotype (IgG (IgG1, IgG2, IgG3, IgG4), IgA (IgA1, IgA2), IgE, IgM, IgD). The fragments of antibodies include in particular the fragments Fab, Fab′, F(ab′)2, Fv, scFv, Fabc or Fab comprising a portion of the Fc region and the single-chain antibody fragments derived from Camelidae or shark immunoglobulins (V_{H}H and V-NAR domain simple antibodies). The derived antibody molecules include polyspecific or multivalent and immunoconjugated antibodies. The multi-specific scFv (dia, tris or tetrabodies), for example scDb (single-chain diabodies) or taFv (tandem scFv fragments) type diabodies, and the minibodies can be given as nonlimiting examples. The mini bodies are in particular scFv-HLX; scFv-ZIP; scFv-CH3, scFv-Fc or other type.

Another aspect of the present invention relates to an isolated polynucleotide coding for a variant conforming to the invention such as defined above. Said polynucleotide is DNA, RNA or a mixture of DNA and RNA, recombinant or synthetic. The DNA sequence may advantageously be modified so that the use of codons is optimal in the host in which it is expressed.

Another aspect of the present invention relates to a vector comprising said polypeptide. Many vectors are known as such; the choice of an appropriate vector depends on the intended use of this vector (for example replication of the sequence of interest, expression of that sequence, maintenance of that sequence in extra-chromosomic form, or else integration in the chromosomal material of the host), and also the nature of the host cell. For example, bare nucleic acids (DNA or RNA) can be used or viral or bacterial vectors. The viral vectors are in particular adenovirus, retrovirus, lentiviruses and the AAV in which the sequence of interest was previously inserted; said sequence (isolated or inserted in a plasmid vector) can also be associated with a substance allowing it to cross the membrane of the host cells, such as a transporter like a nanotransporter or liposome preparation, or cationic polymers or else inserted in said host cell by using physical methods such as electroporation or microinjection. Further, these methods can advantageously be combined, for example by using electroporation together with liposomes.

Preferably, said vector is an expression vector comprising all the elements necessary to the expression of the variant such as defined above. For example, said vector comprises an expression cassette including at least one polynucleotide such as defined above, under the control of appropriate transcription and possibly translation regulating sequences (promoter, activator, intron, initiation codon (ATG), stop codon, polyadenylation signal, splice site), in order to allow the expression of the variant conforming to the invention in a single host cell.

Another aspect of the present invention relates to a prokaryotic or eukaryotic host cell modified by a polynucleotide or a vector conforming to the invention is described above, where the cell can be stably or temporarily modified.

Another aspect of the present invention relates to a pharmaceutical composition comprising at least one variant, polynucleotide, vector and/or cell derived such as defined above and a pharmaceutically acceptable vehicle and/or a carrier substance.

The pharmaceutically acceptable vehicles and the carrier substances are those conventionally used.

The carrier substances are advantageously selected from the group consisting of: unilamellar or multilamellar liposomes, ISCOM, virosomes, viral pseudo-particles, saponin micelles, solid microspheres of saccharide (poly(lactide-co-glycolide)) or auriferous nature, and nanoparticles.

The pharmaceutical composition may further comprise at least one therapeutic agent, in particular anti-inflammatory or immunomodulating.

The pharmaceutical composition comprises a therapeutically active quantity of variant, polynucleotide, vector and cell. A therapeutically active quantity means a sufficient dose for producing a therapeutic effect on the disease to be treated, meaning reducing the symptoms of this illness. This effective dose is determined and adjusted as a function of factors such as age, gender and weight of the subject. The pharmaceutical composition according to the invention comes in a form for delivery suited to the chosen administration. The composition is generally administered according to the usual immunotherapy protocols at doses and for sufficient time in order to induce an effective response against the pathology to be treated. The administration may be subcutaneous, intramuscular, intravenous, in particular by infusion, intradermal, intraperitoneal, oral, sublingual, rectal, vaginal, intranasal, by inhalation or by transdermal application. The composition comes in a form for delivery suited to a selected administration.

The pharmaceutical composition according to the present invention is used in immunotherapy in the treatment of inflammatory or autoimmune pathologies. It may be used in combination with other therapeutic or surgical treatments, in particular in combination with other therapeutic agents such as defined above, where the composition according to the invention and the other therapeutic agents may be administered simultaneously, separately or sequentially.

The inflammatory or autoimmune pathologies are those which are conventionally treated with anti-TNF alpha. Ankylosing spondylitis, rheumatoid arthritis, hemorrhagic colitis, psoriatic arthritis, Crohn's disease, cutaneous psoriasis and juvenile arthritis can be given as nonlimiting examples of these pathologies.

The present invention also relates to a derived variant, polynucleotide, vector and/or cell such as defined above for use as medication, in particular in immunotherapy, in the treatment of autoimmune or inflammatory pathologies such as defined above.

An object of the present invention is also an immunotherapy method, in particular intended for the treatment of inflammatory or autoimmune pathologies such as defined above, characterized in that it comprises the administration to an individual of an effective dose of the derived variant, polynucleotide, vector and/or cell conforming to the invention such as defined above by any appropriate means such as defined above. Preferably, the method comprises the administration of a pharmaceutical composition according to the invention such as defined above.

The polynucleotides according to the invention are obtained by conventional methods, well known in themselves. For example, they can be obtained by amplification of a nucleic sequence by PCR or RT-PCR or else by complete or partial chemical synthesis. The eukaryotic or prokaryotic expression recombinant vectors are built and inserted in host cells by conventional methods of recombinant DNA or genetic engineering, which are well known in themselves. In particular it involves expression vectors conventionally used for the production of antibodies, in particular human or humanized therapeutic antibodies such as tandem type vectors allowing the simultaneous expression of heavy and light chain antibodies. The variants produced by the host cells modified by the recombinant vector are purified by conventional methods for purification of immunoglobulins, in particular by affinity chromatography.

The features disclosed in the preceding paragraphs may, optionally, be put into practice. They may be put into practice independently of each other or in combination with the others.

### BRIEF DESCRIPTION OF THE DRAWINGS

Other characteristics, details and advantages of the invention will appear to the reader of the following detailed description which refers to nonlimiting examples showing the identification and characterization of the variants according to the present invention, and also to the attached figures, on which:

FIG. 1 shows the sequences of the variable parts of adalimumab. The sequences are numbered according to the Kabat nomenclature. The non-germinal residues designate the mutations relative to the alleles having the greatest homology. The residues involved in the direct interaction with TNF alpha come from the structure published in Hu et al. J. Biol. Chem. 288, 27059-27067 (2013). The heavy-chain variable domain (VH) corresponds to the sequence SEQ ID NO: 1 and the light-chain variable domain (VL) corresponds to the sequence SEQ ID NO: 2.

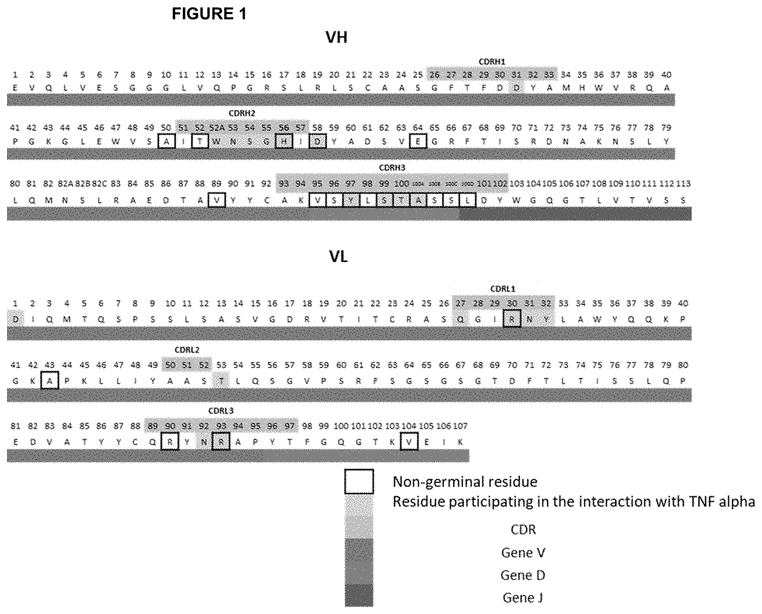

FIG. 2 shows the T cell epitopes of adalimumab and predicted interaction cores. The T cell epitopes of adalimumab were identified in vitro by activation test of the T lymphocytes (data from Meunier et al.). They are principally located on the CDR2 and CDR3 of the heavy chain. An isolated epitope was also identified on the light chain near CDR3. The possible interaction cores were identified by the netMHCllpan algorithm in gray. The major cores, because they were predicted by a significant number of alleles, are shown in dark gray. Sequence of positions P41 to F67 including the CDRH2 (SEQ ID NO: 5); Sequence of positions N82A to W103 including the CDRH3 (SEQ ID NO: 6); Sequence of positions L73 to Q100 including the CDRL3 (SEQ ID NO: 7).

FIG. 3 shows the prediction of the substitutions reducing the immunogenicity scores for the interaction with the HLA II molecules. The netMHCllpan prediction algorithm is used in parallel in order to predict the average effect of each substitution on the binding of the peptides with the HLA class II molecules. A panel of 46 alleles of HLA II molecules covering 80% of the global population was used ([Table 1]) and an immunogenicity threshold of 20%. The mutations lowering the immunogenicity scores are indicated with gray shading (darker=lower predicted immunogenicity). Sequence of positions E46 to E64 overlapping the CDRH2 (SEQ ID NO: 8); Sequence of positions V89 to G104 overlapping the CDRH3 (SEQ ID NO: 9).

FIG. 4 shows the expression and selection of Fab libraries by Yeast Surface Display. A: The expression cassette is composed of a Gal1/Gal10 bidirectional promoter with on either side the heavy chain and the light chain of the antibody associated with the Aga2 signal peptide. The heavy chain is fused in the Aga2P domain. B: The expression of the antibody is analyzed by means of an APC coupled antibody directed against the Ck domain and the interaction is measured by a PE coupled streptavidin. The DMS libraries are sorted by rectangular windows based solely on the PE fluorescence.

FIG. 5 shows by Deep Mutations Scanning the substitutions of CDRH2 and CDRH3 not having any impact on the function of the adalimumab. CDRH2 and CDRH3 permittivity matrices derived from positively sorted populations. The mutants that have an enrichment greater or equal to the parental sequence are indicated with gray shading. The darker the gray, the greater the enrichment, indicating a good affinity for TNF alpha. Sequence of positions E46 to E64 overlapping the CDRH2 (SEQ ID NO: 8); Sequence of positions V89 to G104 overlapping the CDRH3 (SEQ ID NO: 9).

FIG. 6 shows the substitutions of CDRH2 and CDRH3 combining lower predicted immunogenicity and maintaining functionality (DMS), corresponding to the combination of FIGS. 3 and 5. Sequence of positions E46 to E64 overlapping the CDRH2 (SEQ ID NO: 8); Sequence of positions V89 to G104 overlapping the CDRH3 (SEQ ID NO: 9).

FIG. 7 shows the combinatorial libraries for de-immunization. The major interaction cores and their anchoring positions are shown in gray. The libraries were designed so as to be located on these interaction cores. For the library covering CDRH2, the indicated degenerate codons were used. For the library covering CDRH3, the corresponding mix of three nucleotides for each of the positions on the indicated amino acid panel was used. Sequence of positions P41 to F67 including the CDRH2 (SEQ ID NO: 5); Sequence of positions N82A to W103 including the CDRH3 (SEQ ID NO: 6).

FIG. 8 shows the screening of the combinatorial libraries by FACS. The two combinatorial libraries covering CDRH2 and CDRH3 were screened independently. The first equilibrium selection phase comprises three successive sortings at decreasing concentrations of biotinylated TNF alpha. The first sorting at 3 nM and the last at 500 pM are shown in this figure. The second selection phase consists of selecting the mutant by the dissociation speed thereof. In order to do that, the libraries are incubated 3 hours with biotinylated TNF alpha and then placed in competition for 24 hours with non-biotinylated TNF alpha. At the end of 24 hours, the libraries were sorted in order to select the mutants for which the dissociation speed is the slowest.

FIG. 9 presents the progression of the diversity of amino acids during different selection steps. The diversity of amino acids for the zone CDRH2 (A) is shown before and after the equilibrium and kinetic selection steps. For CDRH3 (B), the diversity is shown at the end of the first magnetic selection step and then after the equilibrium and kinetic selection steps. The amino acids are shown as a function of their percentage in a sample of 1000 sequences. The native amino acid is shown in gray and the substitutions in black. Sequence of positions E46 to A60 including the CDRH2 (SEQ ID NO: 10); Sequence of positions D86 to S100C including the CDRH3 (SEQ ID NO: 11).

FIG. 10 shows the screening of the CDRH2-CDRH3 combinatorial library. The first equilibrium selection phase comprises three successive sortings at decreasing concentrations of biotinylated TNF alpha. The first sorting at 3 nM and the last at 500 pM are shown in this figure. The second selection phase consists of selecting the mutant by the dissociation speed thereof. In order to do that, the library is incubated 3 hours with biotinylated TNF alpha and then placed in competition for 24 hours with non-biotinylated TNF alpha. At the end of 24 hours, the library is sorted in order to select the mutants for which the dissociation speed is the slowest.

FIG. 11 shows the analysis of the immunogenic potential of the mutants of interest. A: Prediction by netMHCIIpan of the probability of interaction with the HLA II molecules at a 20% threshold for the selected mutants and the native sequence. The predictions are given independently for the zone of CDRH2 and that of CDRH3. B: Sequence of mutated cores (mutation in red) and effect of these various mutations on the prediction for these cores. ITWNSGHID (SEQ ID NO: 12); INWNGGHRD (SEQ ID NO: 13); INWNGGHSD (SEQ ID NO: 14); YYCAKVSYL (SEQ ID NO: 15); VSYLSTASS (SEQ ID NO: 16); YLSTASSLD (SEQ ID NO: 17); YYCAKSQYL (SEQ ID NO: 18); YYCAKTTYL (SEQ ID NO: 19); YYCAKAHYL (SEQ ID NO: 20); SQYLSTASS (SEQ ID NO: 21); TTYLSTASS (SEQ ID NO: 22); AHYLSTASS (SEQ ID NO: 23); YLPTASSLD (SEQ ID NO: 33).

FIG. 12 shows the detail from the netMHCllpan prediction for adalimumab and the three mutants of interest

### EXAMPLES

### Materials and Methods

### 1. Construction of the Libraries

The DMS libraries were built by PCR assembly. For each position, a direction primer comprising the NNS degenerate codon was used for randomizing the affected amino acid. At the outcome of the PCR assembly, the mutated genes are purified independently on gel and then regrouped to form a library.

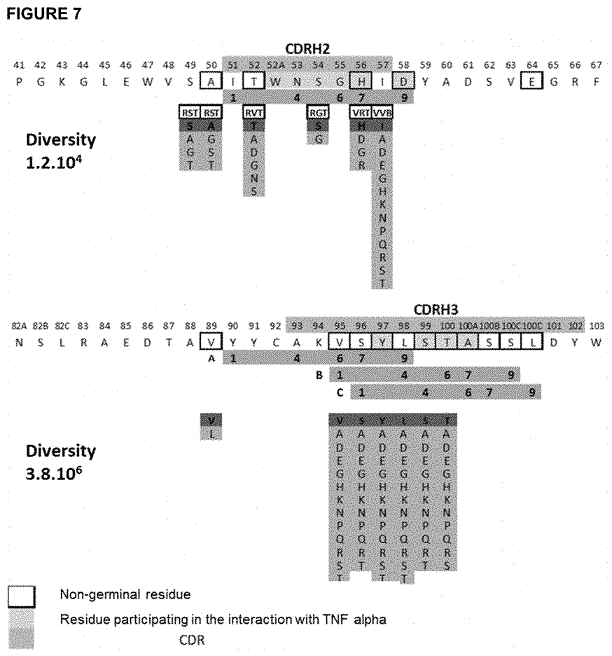

For the de-immunization libraries, PCR assembly was also used. For CDRH2, diversity was inserted by means of degenerate codons chosen with the help of the CodonCalculator tool (http://guinevere.otago.ac.nz/cgi-bin/aef/CodonCalculator.pI) and indicated in FIG. 7. For the CDRH3, a primer synthesized by trinucleotides (Ella Biotech GmbH, Martinsread, Germany) was used in order to insert the intended diversity.

The final de-immunization library was constructed by PCR assembly with plasmids extracted from CDRH2 and CDRH3 libraries at the end of the selection.

### 2. Transformation and Selection by YSD

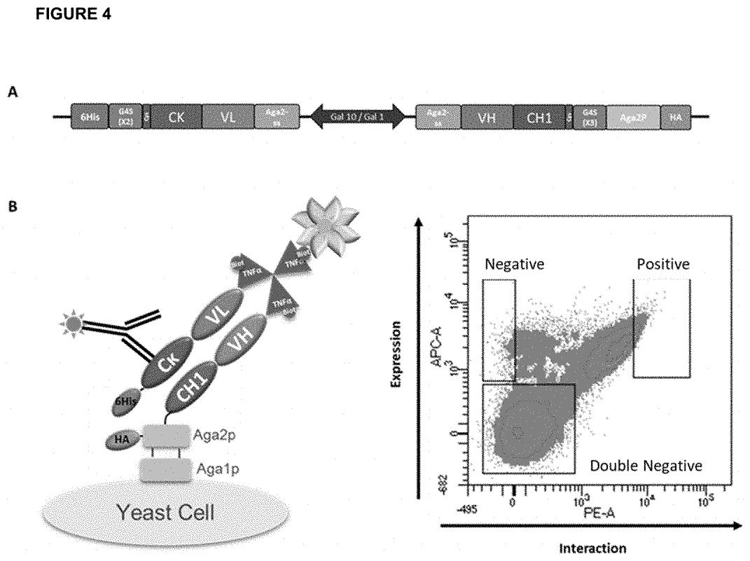

The libraries were cloned in a bicistronic plasmid derived from the plasmid pCT-L7.5.126 (Addgene plasmid #429000) and described in FIG. 4. Like pCT-L7.5.126, it has a CEN/ARS yeast replication origin, a TRP auxotrophy gene, a colE1 bacterial replication origin, an ampicillin resistance gene, and a bidirectional promoter inducible with galactose GLA1/GAL10. The cloning of the library in the YSD expression plasmid was done by homologous recombination during the cellular transformation EBY100 (ATCCÓ MYA-4941TM; a GAL1-AGA1:: URA3 ura3-52 trp1 leu2 D1 his3 D200 pep4:: HIS2 prb1D1.6R can1 GAL) as described in the previous part. For each of the transformations 3 μg of linearized plasmid (NheI and SalI for VH, NcoI and Pfl23II for VL) and 6 μg of insert were used for each of the transformations. All the libraries were generated by electroporation according to the method described by Benatuil et al. Protein Eng. Des. Sel. 23, 155-159 (2010). Each library was transformed in a single reaction (about 5·10⁷ independent clones obtained per reaction) except for the CDRH3 de-immunization library which needed two reactions because of the diversity thereof. The number of transformed clones was determined from a spread with a dilution of 1:1000; a number of transformed clones at least 10 times greater than the size of the library was retained. The libraries thus cloned in the plasmid were cultured in SD-CAA medium [6.7 g/L yeast nitrogen base without casamino acids, 20 g/L dextrose, 5 g/L casamino acids, 100 mM sodium phosphate pH 6.0] and were passed twice before inducing expression in SG-CAA medium [6.7 g/L yeast nitrogen base without casamino acids, 20 g/L galactose, 5 g/L casamino acids, 100 mM sodium phosphate, pH 6.0] in order to minimize double transformants. The culture and expression steps were done according to the description given in the previous part.

The selection of DMS libraries was done in a single step by FACS on an ARIA III device (Becton Dickinson, Franklin Lakes, United States). After induction of the expression, the libraries were incubated 3 hours at 20° C. with biotinylated TNF alpha (ACROBiosystems, Newark, United States) at an 80 pM concentration. After washing the cells with PBS 0.1% BSA, they were marked by using an APC coupled antibody directed against the OK domain (Thermo Fisher Scientific, Waltham, United States; dilution 1:100) and a PE coupled streptavidin (Thermo Fisher Scientific, Waltham, United States; dilution 1:100). The selection of libraries was done by means of rectangular sorting window containing 5% of the clones of interest according to the optimal parameters described by Kowalski et al. PLoS One 10, 1-23 (2015)). The selection of de-immunization libraries was done in several steps. After a possible magnetic sorting by using anti-biotin magnetic beads (Miltenyi Biotec, Bergisch, Germany) after 3 hours of incubation at 20° C. at a 10 nM concentration of biotinylated TNF alpha as described by Chao et al. Nat Protoc 1, 755-768 (2006). The libraries underwent different steps of sorting by FACS. First, three successive steps of equilibrium sorting at decreasing concentrations of biotinylated TNF alpha (3 nM, 1 nM then 500 pM) and then a step of selection by dissociation speed. For each of the sortings, Fab expression was induced and then the cells were incubated for 3 hours at 20° C. with biotinylated TNF alpha before being sorted by FACS. For kinetic sorting based on dissociation speed, the cells were incubated three hours with 20 nM of biotinylated TNF alpha, and then they were washed and incubated for 24 hours with non-biotinylated TNF alpha (Thermo Fisher Scientific, Waltham, United States) before being sorted by FACS.

### 3. NGS Sequencing of Libraries and Analysis of Data

The plasmids were extracted from the cells by enzymatic lysis using the Zymoprep Yeast Plasmid Miniprep II kit (Zymo Research, Irvine, United States). The corresponding fragments were then amplified and the illumina adapters and multiplexing labels added by two PCR steps as described by Kowalsky, C. A. et al. PLoS One 10, 1-23 (2015). The libraries were sequenced in paired-end on a MiSeq using V2 kit 2×150 cycles or on a iSeq still with 2×150 cycles (Illumina, San Diego, United States). For the DMS libraries a minimum sequencing depth of 50× was followed.

The sequences were multiplexed and processed independently on the Galaxy platform (https://usegalaxy.org/) by means functions described by Blankenburg et al., Bioinformatics 26, 1783-1785 (2010). The sequences are unpaired (fasrq-join) and only the sequences having a quality score greater than equal to 30 were retained (FASTQ Quality Trimmer). The sequences were then aligned (Align.seqs) and only the region of interest is retained (Chop.seqs). Finally the sequences are translated (transeq) and the identical sequences are counted and aggregated (Group). The development of the diversity of each of the positions was shown in weblogo form (http://weblogo.threeplusone.com/create.cgi) generated from a thousand sequence sample.

These data were then processed with the R software in order to calculate the frequencies of the various mutants and thus determine their enrichment as follows:

$$Enrichment = \frac{{F}_{output}^{i}}{{F}_{input}^{i}} \qquad [ Math 2 ]$$

Where Fᵢₙₚᵤₜⁱ is the frequency of the mutant i before selection and Fₒᵤₜₚᵤₜⁱ at the outcome of the selection.
For the results of the DMS in matrix form, the mutants are represented by a selective value considering the enrichment of the native sequence:

$$Selective value = {log}_{2} ( \frac{Enrichment}{\frac{{F}_{output}^{wt}}{{F}_{input}^{wt}}} ) \qquad [ Math 3 ]$$

where Fᵢₙₚᵤₜ^{wt} is the frequency of the native sequence before selection and Fₒᵤₜₚᵤₜ^{wt} at the outcome of the selection.

### 4. Prediction of the Peptide/HLA II Molecule Interaction

The predictions of interaction with the HLA II molecules were done by means of the netMHCllpan 3.2 algorithm. (Jensen, K. K. et al., Immunology 154, 394-406 (2018)). Briefly this algorithm predicts the probability of interaction of a sequence with selected HLA II molecules and provides a result for each allele relative to a peptide set. A value of 1% for a peptide means that it is among the 1% of peptides having a high probability of interaction with HLA II molecules. For this study we used a 20% threshold value below which the peptides are considered as immunogenic, thus each nonhuman core below the 20% threshold for an allele counts for one unit in the score. For hitmaps, the scores are given relative to the native sequence; the negative score showing a reduction of the number of peptides below the 20% threshold. For each of the predictions made, it is the panel 46 alleles published by McKinney et al., covering over 80% of the phenotypes for each locus, which was used (McKinney, D. M. et al., Immunogenetics 65, 357-370 (2013)). ([Table 1]).

TABLE 1
Phenotypic and Genotypic Frequency from the Panel of HLA
II Molecules Used (cf. McKinney, D. M. et al., op. cit.)

| | Genotypic | Phenotypic |
|---|---|---|
| Allele(s) | Frequency | Frequency |
| DRB1*0101 | 2.8 | 5.4 |
| DRB1*0301 | 7.1 | 13.7 |
| DRB1*0302 | 1.1 | 2.1 |
| DRB1*0401 | 2.3 | 4.6 |
| DRB1*0402 | 1.1 | 2.2 |
| DRB1*0403 | 2.3 | 4.5 |
| DRB1*0404 | 1.9 | 3.8 |
| DRB1*0405 | 3.1 | 6.2 |
| DRB1*0407 | 2.4 | 4.8 |
| DRB1*0411 | 1.6 | 3.3 |
| DRB1*0701 | 7.0 | 13.5 |
| DRB1*0802 | 2.5 | 4.9 |
| DRB1*0901 | 3.1 | 6.2 |
| DRB1*1101 | 6.1 | 11.8 |
| DRB1*1102 | 1.1 | 2.2 |
| DRB1*1103 | 0.3 | 0.5 |
| DRB1*1104 | 1.4 | 2.8 |
| DRB1*1201 | 2.0 | 3.9 |
| DRB1*1301 | 3.2 | 6.3 |
| DRB1*1302 | 3.9 | 7.7 |
| DRB1*1303 | 1.2 | 2.4 |
| DRB1*1304 | 0.1 | 0.2 |
| DRB1*1401 | 3.4 | 6.7 |
| DRB1*1402 | 2.8 | 5.6 |
| DRB1*1501 | 6.3 | 12.2 |
| DRB1*1601 | 1.0 | 1.9 |
| Total DRB1 | 71.1 | 91.7 |
| DRB3*0101 | 14 | 26.1 |
| DRB3*0202 | 18.9 | 34.3 |
| DRB3*0301 | 6.7 | 13 |
| DRB4*0101 | 23.7 | 41.8 |
| DRB5*0101 | 8.3 | 16 |
| DRB5*′0102 | 5.1 | 9.8 |
| Total DRB3/4/5 | 76.7 | 94.6 |
| DQA1*0501/DQB1*0201 | 5.8 | 11.3 |
| DQA1*0201/DQB1*0201 | 5.7 | 11.1 |
| DQA1*0501/DQB1*0301 | 19.5 | 35.1 |
| DQA1*0301/DQB1*0302 | 10 | 19 |
| DQA1*0401/DQB1*0402 | 6.6 | 12.8 |
| DQA1*0101/DQB1*0501 | 7.6 | 14.6 |
| DQA1*0102/DQB1*0502 | 3.5 | 6.9 |
| DQA1*0102/DQB1*0602 | 7.6 | 14.6 |
| Total DQA1/DQB1 | 66.3 | 88.7 |
| DPA1*0201/DPB1*0101 | 8.4 | 16 |
| DPA1*0103/DPB1*0201 | 9.2 | 17.5 |
| DPA1*0103/DPB1*0401 | 20.1 | 36.2 |
| DPA1*0103/DPB1*0402 | 23.6 | 41.6 |
| DPA1*0202/DPB1*0501 | 11.5 | 21.7 |
| DPA1*0201/DPB1*1401 | 3.8 | 7.4 |
| Total DPB1 | 76.5 | 94.5 |

Further, a set of sequences covering the most frequent human immunoglobulin genes was used in order to evaluate the human or nonhuman character of each of the cores.

### 5. Production and Purification of Fab and IgG

The mutants 1 to 8 and also native adalimumab were produced in Fab format, and the mutants of interest (1, 2 and 7) and adalimumab were also produced in IgG format. The heavy and light variable chains of the antibodies were cloned in the plasmids respectively AbVec2.0-IGHG1 and AbVec1.1-IGKC.²⁴ For the production of Fab, the heavy chain was cloned in a plasma derived from AbVec2.0-IGHG1 from which the domains CH2 and CH3 were withdrawn and replaced by a 6His tag. The production of Fab and IgG was done translationally with HEK293 Freestyle cells (Thermo Fisher Scientific, Waltham, United States) and cultivated in the associated medium. The transfection was done at a density of 2.5·10⁶ cells/mL of culture; a PEI solution (Sigma-Aldrich, Saint-Louis, United States) was used as transfection agent. The plasmids were added to the cultures at a 1:1 ratio and at a final concentration in the culture of 1.5 μg/mL for each plasmid. After 5 minutes of stirring at 37° C. and 8% CO₂, PEI was added drop by drop to a final concentration of 9 μg/mL of culture. After 24 hours under stirring at 37° C. and 8% CO₂, the culture was diluted by half. The production was stopped 7 hours after transfection and the supernatant was recovered by centrifuging at 4° C. for 10 minutes at 3000 G and then 20 minutes at 20,000 G. The proteins were then purified on an AKTA system (GE Healthcare, Pittsburgh, United States). For the Fab, the purification was done by using a HisTrap Excel column (GE Healthcare, Pittsburgh, United States) with elution by an imidazole buffer. Following the purification, the Fab were dialyzed in order to reduce the imidazole concentration. The IgG were purified by means of a HiTrap Protein A HP column (GE Healthcare, Pittsburgh, United States), and then a second time by SEC on a Sephacryl S-200 HR column (GE Healthcare, Pittsburgh, United States), in order to keep the monomeric form of the IgG.

### 6. Measurement of Affinity

The affinity measurements were done kinetically with an Octet Red96 (Molecular Devices, San Jose, United States) according to the protocol described by Schroter et al. (MAbs 7, 138-151 (2015)). Briefly, biotinylated TNF alpha is immobilized on streptavidin sensors (Streptavidin (SA) Biosensor) at a 20 nM concentration. After saturation the sensors in a blocking solution containing 10 μg/mL biotin (Sigma-Aldrich, Saint-Louis, United States), the association and dissociation are measured over 20 and 40 minutes respectively. For the mutants 3 to 6 and the mutant 8, the affinity measurements were done at three Fab concentrations: 10 nM, 5 nM and 2.5 nM, plus a reference at 10 nM without TNFα. For adalimumab and the mutants 1, 2 and 7, the analyses were done for six concentrations of Fab: 15 nM, 10 nM, 5 nM, 2.5 nM, 1.25 nM et 0.625 nM, plus a reference at 15 nM without TNFα. During the analysis, the reference is subtracted from each curve and a 1:1 global Langmuir model is applied in order to get the affinity parameters.

### 7. Analysis of the Mass of the Antibodies

The masses of the IgG products (adalimumab, Mutants 1, 2 and 7) and of the adalimumab in its commercial version (Humira) were determined by mass spectrometry. The analysis is done by a Q-Orbitrap type high-resolution device (Thermo Fisher Scientific, Waltham, United States) by UHPLC-MS as described by Contrepois et al. (J. Proteome Res. 9, 5501-5509 (2010)).

SEC-MALS was done on the GIPSI platform at Université Paris-Sud on an HPLC (Shimadzu) with a Superdex 200 10/300 GL increase column (GE Healthcare, Pittsburgh, United States).

### Example 1: Analysis of the Immunogenic Regions

### Location of the Immunogenic Regions

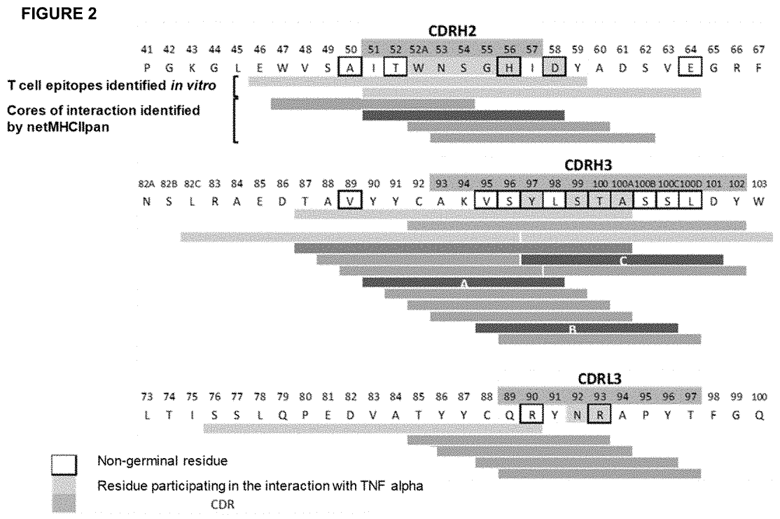

Prior to this work, the adalimumab T cell epitopes were identified in vitro by specific activation tests of the CD4 T lymphocytes by means of peptides overlapping a length of 15 or 20 amino acids. The regions comprising the T cell epitopes are mostly located within the heavy chain, CDR2 over as zone included between the E46 and E64 residues and the CDR3 between the L82c and T107 amino acids (Meunier, S. et al., Cellular & Molecular Immunology, 2019, Oct. 28. doi: 10.1038/s41423-019-0304-3). A T cell epitope is also described on the light chain upstream from the CDR3 (S76 to R90). (FIG. 2) It can also be seen that the identified T cell epitopes all comprise residues different from the germinal line (FIG. 2: bracketed residues) and may consequently be perceived as not belonging to it by the immune system. In order to protect the possible modalities of interaction of these T cell epitopes with the HLA II molecules, the algorithm was used. The algorithm predicts four interaction cores for the epitopes identified in the CDRH2 (in gray on FIG. 2). In light of the MAPPS data derived from Meunier et al., op. cit. ([Table 2]) showing that the overlapping region between the two T cell epitopes of the CDRH2 is systematically retained among the peptides naturally prepared by the dendritic cells, the interaction core is very probably contained in this region.

TABLE 2
Peptides Presented by the HLA II Molecules Obtained by MAPP

| | Epitope | | Number of |
|---|---|---|---|
| Antibody | regions | Sequences | donors |
| HCDR2 | AH46-65 | [symbol] APGKGLEWVSAITWNSGHIDYADSVEGRFTI [symbol] | |
| | | APGKGLEWVSAITWNSGHID | 3 |
| | | WVSAITWNSGHIDYADS | 2 |
| | | VSAITWNSGHIDYADS | 1 |
| | | ITWNSGHIDYADSVEGRFTI | 1 |
| | | NSGHIDYADSVEGRFTI | 1 |
| HFR3 | AF76-95 | [symbol] ISRDNAKNSLYLQMNSLRAEDTAVYY [symbol] | |
| | | ISPDNAKNSLYLQMNSLRAEDTAV | 1 |
| | | ISRDNAKNSLYLQMNSLPAEDTA | 2 |
| | | RDNAKNSLYLQMNSLRAEDTA | 1 |
| | | DNAKNSLYLQMNSLRAEDTA | 2 |
| HCDR3 | AH86-120 | [symbol] LRAEDTAVYYCAKVSYLSTASSLDYWGQGTLVTVSSASTKGP [symbol] | |
| | | AEDTAVYYCAKVSYLSTAS | 1 |
| | | AKVSYLSTASSLDYWGQ | 1 |
| | | WGQGTLVTVSSASTKGP | 1 |
| LCDR2 | AL41-55 | [symbol] KPGKAPKLLIYAASTLQSGVPSR [symbol] | |
| | | KPGKAPKLLIYAASTLQSGVPS | 9 |
| | | APKLLIYAASTLQSGVPS | 1 |

Among the peptides identified by MAPPS, those corresponding to the T cell epitopes identified by cellular test (in bold) are shown in form of overlapping peptide clusters. [symbol]
indicates data missing or illegible when filed

Thus, the 9-mer “ITWNSGHID” (SEQ ID NO:12) comprising the residues 51 to 58 (dark gray in FIG. 2) predicted by netMHCllpan is most likely the interaction core of the T cell epitopes of the CDRH2 among the predicted cores (light gray in FIG. 2). CDRH3 as the greatest density of T cell epitopes; a set of six overlapping T cell epitopes was predicted in this region. The overlapping nature of these epitopes also leaves reason to think that these overlapping zones could be particularly interesting for destabilizing the peptide/HLA II interaction of several T cell epitopes simultaneously. However the identification of the shared interaction core in the zones is less obvious than in the case of CDRH2 because there is no corresponding peptide in the MAPPS analysis. However, three interaction cores “VYYCAKVSYL” (SEQ ID NO: 15), “VSYLSTASS” (SEQ ID NO: 16) et “YLSTASSLD” (SEQ ID NO: 17) are predicted on a greater number of alleles; also it would be considered to be more probable than the other cores predicted by netMHCllpan (in dark gray on FIG. 2). Finally, for the T cell epitope located on the light chain, curiously, none of the predicted cores allowed defining a probable interaction modality. Also, for this T cell epitope, suppression of the interaction with the HLA II molecules has little interest, even more so since it has only one mutation different from the germinal line in R90. A different approach will therefore be adopted for this T cell epitope; since the R90K mutation towards the germinal residue is described as functional in the Humira® patent, this substitution will be done in order to humanize the T cell epitope.

Since the CDRH2 and CDRH3 are major immunogenicity zones found in several donors, action was focused on these two regions in order to get the greatest possible immunogenicity reduction.

### Identification of the Mutants Reducing the Immunogenicity

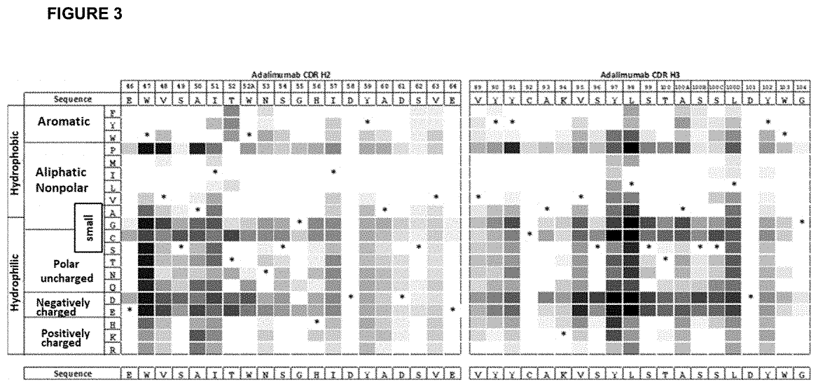

In order to reduce the T cell epitope/HLA II affinity and thus define a de-immunization strategy, the inventors first sought to understand the influence of the substitutions on the prediction from the netMHCllpan algorithm. A systematic approach was adopted for evaluating the influence of all the substitutions possible in the area of the CDRH2 and the CDRH3 on the presentation by the HLA II molecules. The netMHCllpan algorithm was used in parallel in order to predict the average effect of each mutation on a panel of 46 HLA II alleles covering over 80% of the global population. These predictions, shown in matrix form, show the relative effect of the mutation compared to the native sequence (FIG. 3).

The CDRH2 offers many mutations serving to reduce the probability of interactions with HLA II molecules (substitutions colored with gray shading (darker gray=lower predicted immunogenicity, FIG. 3). Substitutions to hydrophilic amino acids and also to proline generally tend to lower the prediction scores over most of the positions. The insertion of hydrophobic amino acids on the other hand generally has the opposite effect. Several positions offer substitutions which could lower the prediction scores, mostly hydrophobic amino acids (e.g. W47, V48, I51) but also some polar amino acid substitutions (e.g. T52E, S49D). The other way around, the mutation of some residue such as E46, G55 and D58 does not seem to offer any interest for reducing the immunogenicity.

The results of the prediction for CDRH3 are fairly similar, the hydrophilic residues are preferred for destabilizing the peptide/HLA II interaction. (FIG. 3) The two hydrophobic residues Y97 and L98, but also of the six residues starting with serine S99, and also the amino acids V89, Y90 and Y91 are predicted for having a particularly interesting effect on the interaction with the HLA II molecules. On the other hand, as for CDRH2, some residues do not show any interest for reducing the immunogenicity, such as the residues C92, D101 and G104.

The modifications proposed by netMHCIIpan are globally substitutions towards the hydrophilic amino acid, with an effect that is that much larger if the native residue is hydrophobic. netMHCIIpan does not consider either the structure or the functionality of the antibody; these predictions are to be put into perspective with the tolerance to the mutation of these amino acids. In fact, elimination of all the hydrophobic amino acids over such important regions as the CDR seems extremely risky for the functionality and also for the structure of the antibody. Further residues belonging to these two CDR have been described for their contribution to the interaction with TNFα (Hu et al. J. Biol. Chem. 288, 27059-27067 (2013). In order to take into account all these functional and structural constraints, a second systematic approach was applied, this time to the function.

### Design, Construction and Screening of the DMS Libraries

In order to identify, among the substitutions proposed by netMHCIIpan, those which make it possible to get functional mutations, the inventors decided to perform a functional study of these two regions as a first step. An approach by systematic mutation, call Deep Mutational Scanning (DMS), was chosen for identifying permissive amino acids within these immunogenic zones. This strategy provides for the functional evaluation of all possible substitution at each position with each variant comprising only one mutation.

The two immunogenic regions were processed in two independent libraries. They each cover one region of 20 residues and they were built by PCR assembly. For each of the 20 residues, diversity was introduced by means of a degenerate NNS codon coding for the set of 20 natural amino acids. Degenerate codons were inserted by independent PCR for each position. The 20 PCR products were mixed in equal molar proportions in order to get a library containing all the single substitutions for each of the 20 positions. The libraries were then cloned in an expression plasmid for the Yeast Surface Display (YSD) allowing the expression in Fab form. (FIG. 4A) The cloning of the two libraries was done independently by homologous recombination during the transformation in yeast. The libraries were next expressed in YSD and the mutants of interest selected by FACS. (FIG. 4B) The screening was done under the conditions described by Kowalsky et al. (PLoS One 10, 1-23 (2015)). The libraries were screened once after incubation for 3 hours at a TNF alpha concentration less than K_{D} of the adalimumab in order to be able to observe both an increase and loss of function. The 5% of the expressed mutants having the lowest signal for interaction were selected (see “Negative” sorting window, FIG. 4C), in order to select the mutants having a harmful impact on the function. Analogously, the 5% of the militants showing the greatest interaction were selected (see “Positive” sorting window, FIG. 4C). The yeast population before any selection step and also the two selected populations were next sequenced by NGS.

### Analysis of the Permissivity of the Two Immunogenic Regions

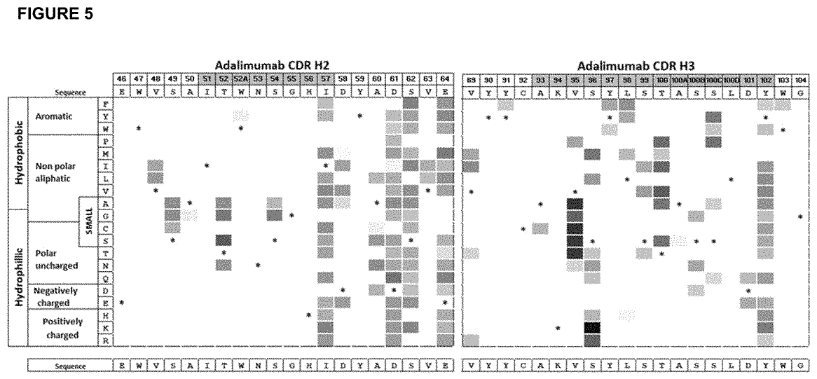

Based on the sequencing data, the enrichments of the parental sequence (wild type) and also that of each of the mutants were calculated. With these it was possible to calculate for each of the variants a score called selective value (i.e. fitness) which corresponds to the base two logarithm of the relative enrichment of the variant compared to the native sequence. In order to get an overall view of the permissivity of each of the regions, the mutants are shown in a matrix with the color proportional to the selective value thereof. For the negative selection, the amplitude of the selective values is from −8 to 3, or enrichment 256 times lower to 8 times greater than the native sequence. For the positive selection, the amplitude of the selective values is substantially the same. The mutants that have an enrichment greater or equal to the parental sequence are indicated with gray shading. The darker the gray, the greater the enrichment, indicating a good affinity for TNF alpha. (FIG. 5).

The results obtained from the selection of mutants expressed but not functional are particularly interesting in order to define the residues participating in the action with the TNF alpha. The population derived from the positive selection is necessary for identification of mutations with which to maintain or increase the affinity. Finally, the combination of these two matrices of results allows identification of the residues that are important for the proper expression of the antibody (negative selective value in the two selection conditions).

For the region of the CDRH2, the negative selection serves to identify fairly clearly a central region of six consecutive residues (T52 to H56) for which the selective values are globally higher. This patch of six residues is supplemented by alanine in position 50 and aspartic acid in position 58 on each side. These eight residues, particularly enriched in the negative selection, therefore seem particularly important for the interaction. With the positive selection however, some functional mutations for these residues can be identified, in particular for small size residues (alanine, lysine or serine) T52 and S54. (FIG. 5) From each side of this interaction zone, it is also noted that the ends of the library are less enriched in the negative selection and behave differently in the positive selection. The mutants from the N-terminal part are poorly represented in the positive selection; also these residues seem somewhat involved in the folding and expression of the antibody. The mutants belonging to the C-terminal par, on the other hand, are a little more enriched in the positive selection. (FIG. 5) Many mutations are allowed, including to residues with different properties, all while retaining a selective value equal to or superior than that of the native antibody.

The residues involved in the interaction in the area of CDRH3 seem to be broken into two groups. The residues K94 and V95 on one side and the residues S99 to S100c a little farther. Fairly surprisingly and the opposite of the CDRH2, some of these residues are permissive for nonconservative substitutions. (FIG. 5) It is in particular the case in the positive sorting of the residue V95 for which the selected value is increased when it is substituted by a small amino acid, likewise the mutation of the residue T100 to nonpolar aliphatic residues is seen by an increase of the selected value. The positive selection also reveals that the residues Y90 to K94 are not permissive even though they do not seem to be directly involved in the interaction with TNF alpha. This type of profile is also found for the residues A100A, W103 and G104.

These first libraries serve to identify substitutions allowing retention of the functionality for these two regions of interest. They also serve to show the permissivity difference of the residues from CDRH2 and CDRH3, in particular those directly involved in the interaction. These data were next used in order to design combinatorial libraries with the objective of eliminating the T cell epitopes identified in these zones, while also assuring that they contain functional mutants.

### Example 2: Generation of Adalimumab Mutants with Reduced Immunogenic Potential

### Design of the Libraries

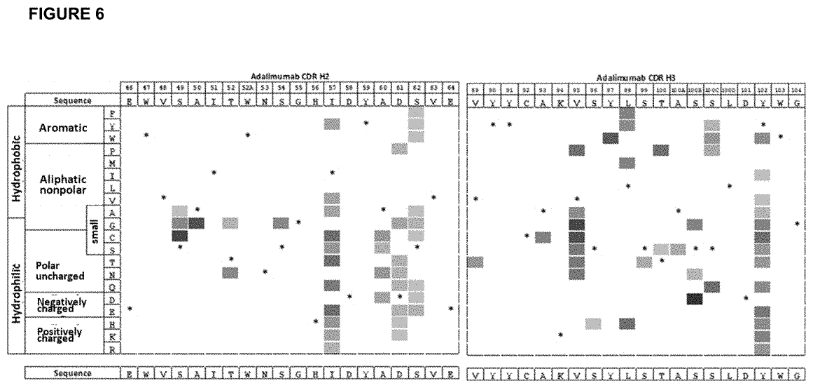

The CDRH2 comprises two overlapping T cell epitopes which most likely share the interaction core “ITWNSGHID” (in dark gray in FIG. 2). Consequently, the mutation strategy is concentrated on this zone. The crosschecking of the matrices derived from the DMS with the prediction by netMHCllpan made it possible to select mutations with which to both destabilize the interaction with the HLA II molecules while also conserving the functionality of the antibody (FIG. 6). The residues S49, A50, T52, S54, H56 and I57 were selected because they all have one or more mutations serving to destabilize the interaction with the HLA II molecules. For each of these residues, a degenerate codon serving to best represent substitutions of interest was chosen. (FIG. 7) For each position the substitution number is reduced (1 to 5) with the exception of the residue I57 which is particularly interesting for reducing the immunogenicity and also very permissive. Thus, this residue was substituted by all the non-hydrophobic amino acids except for cysteine.

The CDRH3 comprises a set of five overlapping T cell epitopes for which we defined a set of three probable interaction cores (in dark gray in FIG. 2). We chose to direct the mutagenesis towards the overlap zone between the three most strongly predicted cores, specifically the residues V95 and T100. In fact, these positions combine many substitutions for which a drop in immunogenicity is expected. Although relatively unpermissive, this region combines amino acids from CDRH3 like valine 95, serine 96, threonine 100 for which various substitutions seem to be tolerated. To maximize the probability of identifying active and potentially de-immunized mutants, we chose to insert in this zone of six consecutive residues all the substitutions over these six consecutive residues by one hydrophilic and/or small size amino acid except for cysteine. This combinatorial strategy could potentially show compensation effects, thus making possible the presence of two substitutions not individually identified. Further, the substitution V89L, serving to germinalize the N-terminal part of the CDRH3, completes this library. (FIG. 7) Because of the large number of combinations, the construction of this library with degenerate codons would have produced a library with too much diversity (>10⁷) for screening in YSD. For this reason, during the synthesis of the primers used for the construction of this library trinucleotide codons were included in order to reduce the diversity to under 10 million mutants.

### Construction and Screening

Each of the libraries was built by PCR assembly, with primers comprising degenerate codons for the CDRH2 and primers synthesized from mixtures of trinucleotides for the CDRH3. These libraries respectively have a diversity of 1.2×10⁴ for CDRH2 and 3.8×10⁶ for CDRH3. As before, they were cloned by homologous recombination in a plasmid for expression and screening in YSD. After transformation of the yeasts and induction of expression, the screening by FACS was done independently for the two libraries and each of the libraries underwent various selection steps.

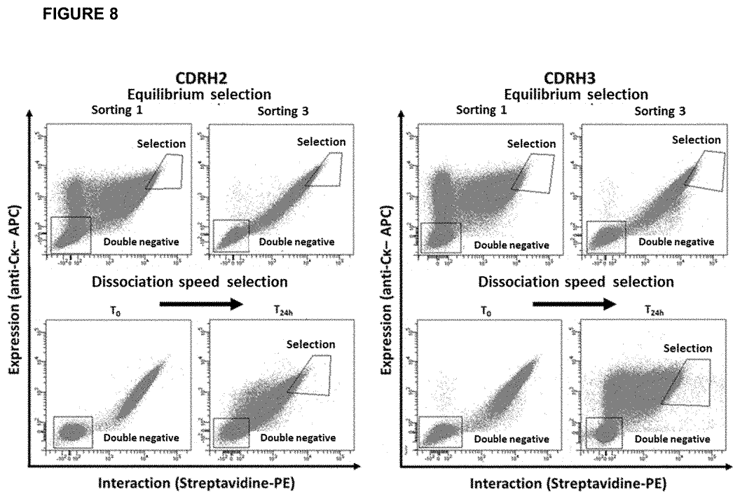

Because of the significant diversity thereof, the library covering the CDRH3 made a direct selection by FACS difficult. This was therefore enriched a first time by magnetic sorting (MACS) with a 10 nM concentration of biotinylated TNF alpha. Except for this enrichment, the screening steps are the same for the two libraries. A first phase of three successive selections at equilibrium was done at decreasing concentrations of biotinylated TNF alpha. (FIG. 8) At the end of these steps, the libraries were selected one last time kinetically in order to get mutants having a slow dissociation speed. To do that, the libraries were saturated for 3 hours with 20 nM biotinylated TNF alpha and then sorted at the end of a 24 hour competition against non-biotinylated TNF alpha. During each of the selections steps, 2 to 5% of the cells having the strongest PE signal were selected by FACS by means of diagonal sorting windows in order to normalize the binding signal by the expression. (FIG. 8)

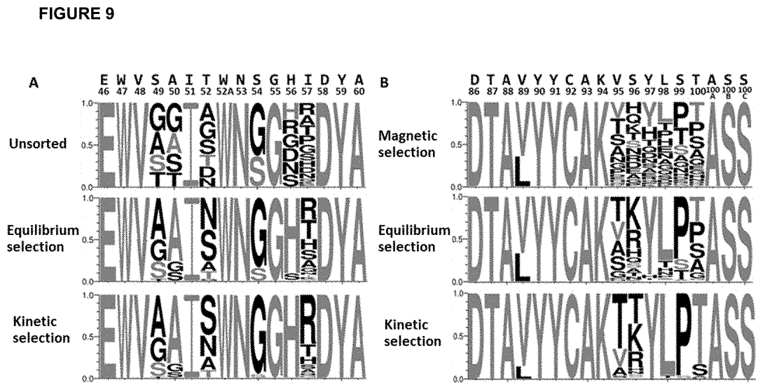

In order to understand the progression of the molecular diversity of the libraries during the selection steps, we chose to do a NGS sequencing after each of the selection steps. The sequencing data from the library covering the CDRH2 show that all the mutations were present in the library before starting screening. (FIG. 8A) At the end of the three selection steps at equilibrium, a return to the native residue for some positions was seen, such as for the residues H56 and A50. In contrast, for the other mutated position several residues different from the native amino acid are found. The last kinetic selection step on the dissociation speed somewhat reduces the diversity of possible substitutions while overall reinforcing the initially observed profiles. At the end of the screening process, among the six targeted residues in the library, the residues S49, T52, S54 and I57 are largely substituted (FIG. 9A). These mutations are for the most part conservative except for the residue I57 for which the most abundant substitutions are hydrophilic amino acids.

The sequencing data allowed us to calculate the enrichment values for each of the mutants at the end of the selection process. The 30 most enriched mutants in this library are shown in [Table 3].

TABLE 3
Selection of the 30 Most Enriched Mutants
derived from the Library Relating to CDRH2

| | | | | | | | | | | | netMHCIIpan | |
|---|---|---|---|---|---|---|---|---|---|---|---|---|
| CDRH2 | | | | | | | | | | Enrichment | rank | |
| A | A | I | N | W | N | G | G | H | R | 417 | 101 | |
| A | A | I | S | W | N | G | G | H | R | 382 | 128 | |
| G | A | I | S | W | N | G | G | H | R | 279 | 97 | |
| G | A | I | N | W | N | G | G | H | R | 222 | 27 | |
| A | A | I | T | W | N | G | G | H | R | 213 | 93 | |
| S | A | I | S | W | N | G | G | H | R | 207 | 124 | |
| A | A | I | A | W | N | G | G | H | R | 197 | 168 | |
| A | A | I | S | W | N | G | G | H | T | 193 | 119 | |
| A | A | I | S | W | N | G | G | H | Q | 192 | 117 | |
| A | A | I | S | W | N | G | G | H | H | 164 | 118 | |
| A | A | I | A | W | N | G | G | H | T | 144 | 161 | |
| G | A | I | S | W | N | G | G | H | T | 133 | 85 | |
| A | A | I | N | W | N | G | G | H | H | 132 | 60 | |
| S | S | I | A | W | N | G | G | H | W | 128 | 142 | |
| A | A | I | N | W | N | S | G | H | R | 122 | 172 | |
| A | A | I | A | W | N | G | G | H | H | 119 | 163 | |
| A | A | I | A | W | N | G | G | H | Q | 109 | 162 | |
| A | A | I | N | W | N | S | G | H | Q | 105 | 148 | |
| S | A | I | S | W | N | G | G | H | Q | 104 | 107 | |
| S | A | I | S | W | N | G | G | H | T | 100 | 109 | |
| A | A | I | A | W | N | G | G | H | S | 93 | 164 | |
| A | A | I | T | W | N | G | G | H | T | 91 | 44 | |
| S | A | I | S | W | N | G | G | H | H | 87 | 110 | |
| A | A | I | S | W | N | G | G | H | S | 82 | 120 | |
| S | A | I | N | W | N | G | G | H | R | 79 | 99 | |
| A | A | I | S | W | N | G | G | H | A | 75 | 131 | |
| S | A | I | T | W | N | G | G | H | R | 71 | 95 | |
| G | A | I | S | W | N | G | G | H | H | 66 | 84 | |
| G | A | I | T | W | N | G | G | H | R | 64 | 94 | |
| G | A | I | N | W | N | G | G | H | T | 62 | 8 | |
| S | A | I | T | W | N | S | G | H | I | 4 | 159 | Adalimumab |

It can be seen that the best mutants were enriched over 200 times; in total after complete screening, 174 mutants had a greater enrichment than that of adalimumab. The netMHCllpan algorithm was once again used in order to rank these mutants and 158 of them are predicted for potentially being less immunogenic.

For the library covering the CDRH3, the sequencing of the population after the first selection by magnetic screening revealed a high diversity on the residues 95 to 100 validating the construction of the library. (FIG. 9B) At the outcome of the selections at equilibrium, the residues Y97 and L98 are strongly retained whereas the other residues have fairly diversified profiles with however a strong dominance for proline in position 99. The last selection step on the dissociation speed (Koff) serves to even better discriminate the mutants according to the TNF alpha binding capacity thereof. The selection by FACS shows in fact, for the library covering the CDRH3, a great heterogeneity of the binding properties of the mutants after 24 hours of dissociation and presence of non-biotinylated TNF alpha. (FIG. 8) This observation is seen, at the end of the sequencing of the selected mutants, by a strong evolution of the profiles of some positions. Positions 89 and 100 undergo a major enrichment towards the native amino acid and the positions 95 and 96 see their diversity reduced. For the positions 97 to 98, the trends observed at the outcome of the first selections are greatly reinforced and the diversity of these positions is very reduced. In the same way as for CDRH2, at the end of the selections only some positions seem to be able to be substituted. These mutations are often conservative with the exception of the residue V95 allowing threonine and alanine and the residue S99 principally mutated in proline. The 30 most enriched mutants in this library are shown in [Table 4].

TABLE 4
Selection of the 30 Most Enriched Mutants derived from the Library Relating to CDRH3

| | | | | | | | | | | | | | netMHCHpan | |
|---|---|---|---|---|---|---|---|---|---|---|---|---|---|---|
| CDRH3 | | | | | | | | | | | | Enrichment | rank | |
| V | Y | Y | C | A | K | T | T | Y | L | P | T | 1.29E+06 | 109 | |
| V | Y | Y | C | A | K | T | K | Y | L | P | T | 1.06E+06 | 168 | |
| V | Y | Y | C | A | K | T | R | Y | L | P | T | 7.07E+05 | 221 | |
| L | Y | Y | C | A | K | T | N | Y | L | P | T | 4.48E+05 | 203 | |
| V | Y | Y | C | A | K | T | S | Y | L | P | S | 3.71E+05 | 116 | |
| L | Y | Y | C | A | K | V | K | Y | L | P | T | 2.51E+05 | 270 | |
| V | Y | Y | C | A | K | V | T | Y | L | P | S | 2.41E+05 | 256 | |
| V | Y | Y | C | A | K | V | K | Y | L | P | T | 2.41E+05 | 264 | |
| L | Y | Y | C | A | K | V | K | Y | L | P | S | 1.88E+05 | 294 | |
| V | Y | Y | C | A | K | A | K | Y | L | P | T | 1.63E+05 | 202 | |
| L | Y | Y | C | A | K | T | T | Y | L | P | S | 1.51E+05 | 158 | |
| V | Y | Y | C | A | K | T | T | Y | L | P | S | 1.49E+05 | 130 | |
| V | Y | Y | C | A | K | A | H | H | L | P | T | 1.22E+05 | 171 | |
| L | Y | Y | C | A | K | V | K | Y | T | P | T | 1.10E+05 | 123 | |
| V | Y | Y | C | A | K | S | K | Y | L | P | T | 1.05E+05 | 194 | |
| V | Y | Y | C | A | K | T | G | Y | L | P | T | 1.00E+05 | 91 | |
| V | Y | Y | C | A | K | V | T | Y | T | P | S | 9.92E+04 | 57 | |
| L | Y | Y | C | A | K | T | K | Y | L | P | T | 9.74E+04 | 177 | |
| V | Y | Y | C | A | K | T | K | Y | L | P | S | 8.71E+04 | 184 | |
| V | Y | Y | C | A | K | V | K | Y | L | P | A | 8.14E+04 | 292 | |
| V | Y | Y | C | A | K | T | R | Y | T | P | T | 8.08E+04 | 33 | |
| V | Y | Y | C | A | K | V | K | Y | L | P | S | 7.19E+04 | 287 | |
| L | Y | Y | C | A | K | T | K | Y | L | P | S | 6.54E+04 | 192 | |
| L | Y | Y | C | A | K | T | R | Y | L | P | S | 6.35E+04 | 244 | |
| V | Y | Y | C | A | K | V | A | Y | L | P | S | 6.20E+04 | 285 | |
| V | Y | Y | C | A | K | V | K | Y | L | P | P | 6.16E+04 | 97 | |
| V | Y | Y | C | A | K | T | H | Y | L | P | T | 6.12E+04 | 125 | |
| L | Y | Y | C | A | K | T | R | Y | L | P | T | 5.94E+04 | 225 | |
| L | Y | Y | C | A | K | T | T | Y | L | P | T | 5.41E+04 | 114 | |
| V | Y | Y | C | A | K | T | N | Y | L | P | T | 5.19E+04 | 163 | |
| V | Y | Y | C | A | K | V | S | Y | L | S | T | 2.55E+02 | 283 | Adalimumab |

It is also observed that the enrichment factors are higher for CDRH2, with values which can reach several million. After screening, the library comprises 310 mutants more enriched then the adalimumab native sequence and, as for CDRH2, a major part of them (282) have a reduced immunogenic potential according to netMHCIIpan.

At the end of selection of these two libraries, a large number of alternative sequences, potentially less immunogenic, resulted both for CDRH2 and CDRH3. These were combined in order to get an entirely de-immunized heavy chain.

### Construction of a Library by Recombination of Selected Mutants

In order to avoid the combination of incompatible mutations on CDRH2 and CDRH3, the choice was made to recombine all of the sequences contained in the two libraries following screening thereof. The sequences of the mutants from each of the libraries were extracted by PCR on the final populations after kinetic sorting. The combination of sequences was then done by random recombination via an assembly by PCR. This combined CDRH2+CDRH3 library was then cloned in the expression plasmid in YSD described above by homologous recombination during the transformation in yeast. The CDRH2+CDRH3 libraries respectively comprising a minimum of 489 and 234 mutants (found at least 10 times during sequencing) at the end of selection thereof, this combinatorial library contains a minimum of 10⁵ variants. This library also incorporates the R90K substitution described in the Humira® patent as preserving the affinity and serving to germinalize the sequence and the CDRH3 region and in that way to remove a minor epitope (Salfed, J. G. et al. Human antibodies that bond human TNFa. (U.S. Pat. No. 6,258,562 B1)).

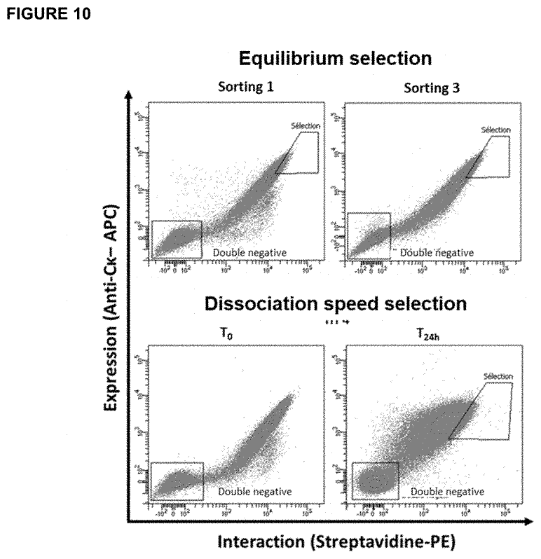

This library was screened according to the same process applied to the previous libraries; specifically three equilibrium sortings with increasing TNF alpha concentrations followed by a selection on the dissociation speed. (FIG. 10) Since this library comes from recombination of already selected sequences, a high initial enrichment in functional mutants is seen even before the first selection step. From the first selection steps, the population is already very homogeneous; screening on the dissociation speed done next however reveals a more heterogeneous population.

### Identification of Clones of Interest

After sequencing, the selected mutants were evaluated according to the enrichment thereof and 245 of them showed a value greater than the native sequence. Among them, over 200 mutants are predicted to have fewer interaction cores with the HLA II molecules than adalimumab. Their sequences however show a redundancy in their sequences, particularly in the area of CDRH3. ([Table 5]).

TABLE 5
Selection of 30 mutants best ranked by netMHCHll pan and
also eight mutants of interest derived from the CDRH2 and CDRH3
recombinant library

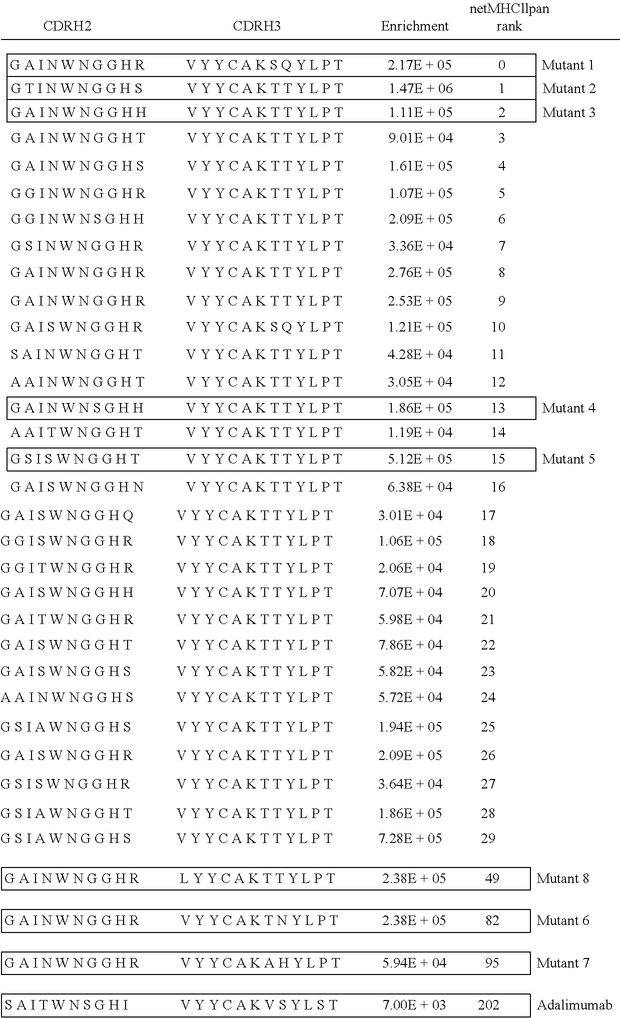

At the outcome of the selection steps, a reduced number of variants of interest were selected in order to carry out a complete biochemical characterization. The mutants were selected for their enrichment better than the native sequence and for their reduced immunogenic potential according to netMHCII pan but also by giving specific importance to selecting mutants with diverse sequences. Based on these criteria, eight mutants were selected to be characterized. ([Table 6]).

### Characterization of Clones of Interest

These mutants and also adalimumab were produced in Fab form in HEK cells in order to be characterized. The affinity of these mutants for TNF alpha was evaluated by Bio Layer Interferometry, all showed a greater affinity than that of adalimumab. ([Table 6])

TABLE 6
Mutants of interest selected in the CDRH2-CDRH3
combinatorial library

| | | | [symbol] | K_{d} | K_{D} | |
|---|---|---|---|---|---|---|
| Antibody | CDRH2 | CDRH3 | [symbol] | [symbol] | (pM) | Enrichment |
| Adalimumab | SAITWNSGHI | V-VSYLST | 39.8(±0.05) | 170(±0.18) | 428 | 7.00E-03 |
| Mutant 1 | GAINWNGGHR | V-SQYLPT | 43.9(±0.03) | 152(±<0.1) | 345 | 2.17E+05 |
| Mutant 2 | GTINWNGGHS | V-TTYLPT | 64.0(±0.07) | 16.5(±0.1) | 25.8 | 1.47E+06 |
| Mutant 3 | GAINWNGGHH | V-TTYLPT | 111(±0.07) | 50.2(±0.12) | 45.2 | 1.11E+05 |
| Mutant 4 | GAINWNSGHH | V-TTYLPT | 64.7(±0.03) | 44.1(±0.10) | 68.1 | 1.86E+05 |
| Mutant S | GDISWNGGHT | V-TTYLPT | 91.7(±0.06) | 8.25(±0.12) | 9.0 | 5.12E+05 |
| Mutant 6 | GAINWNGGHR | V-TNYLPT | 99.8(±0.06) | 40.3(±0.10) | 40.4 | 2.38E+05 |
| Mutant 7 | GAINWNGGHR | V-AHYLPT | 134(±0.01) | 136(±0.10) | 102 | 5.94E+04 |
| Mutant 8 | GAINWNGGHR | L-TTYLPT | 106(±0.07) | 45.5(±0.12) | 42.8 | 2.38E+05 |

[symbol]

indicates data missing or illegible when filed

For some, like the mutants 2 and 5, an affinity increased more than 10 times was measured. The increase of the affinity for the various clones is mostly due to a reduction of the dissociation speed which was a parameter selected during screening. While all these mutants have an enrichment greater than the native antibody, these values do not however always seem rigorously proportional to the affinities measured for these mutants.

The mutants 1, 2 and 7 were selected for the predicted reduced immunogenicity thereof in order to move the characterization forward. These mutants and also the native antibody were produced in HEK and purified to IgG format. An analysis by Orbitrap mass spectrometry served to confirm that the antibodies have a mass corresponding to that expected, with a resolution of order one Dalton. Additionally, an analysis by SEC-MALS (Size Exclusion Chromatography—Multi-Angle Light Scattering) also serve to confirm the monomeric nature of these antibodies.

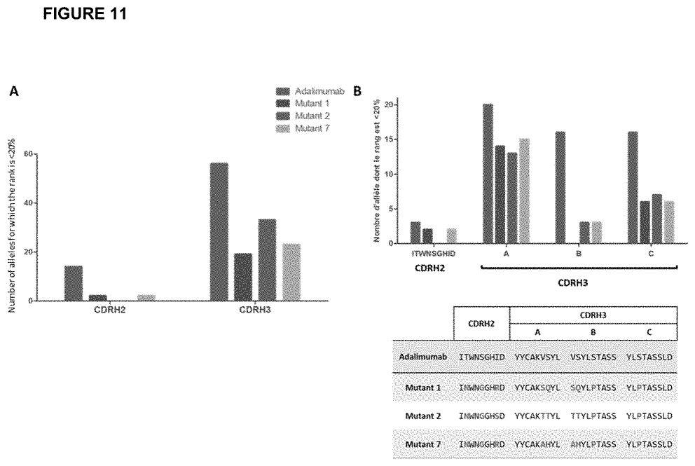

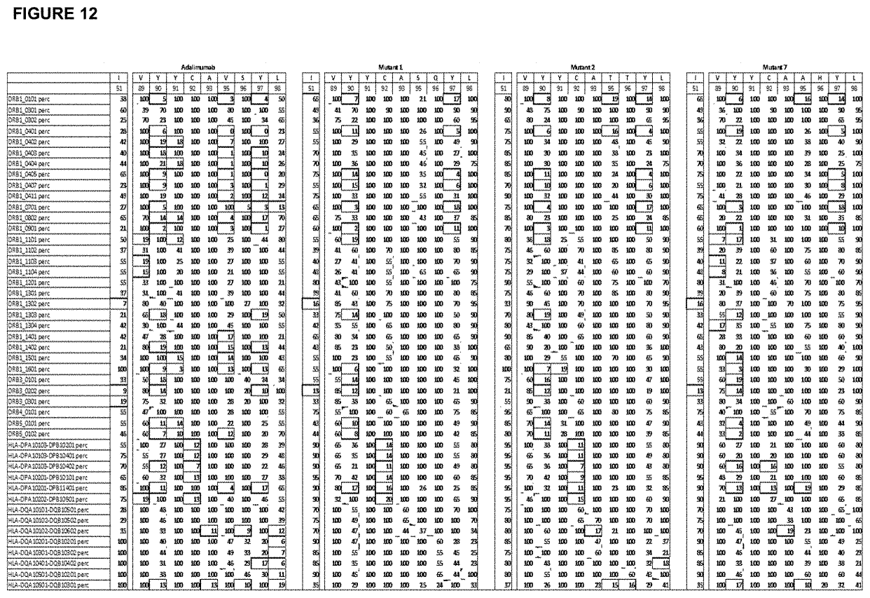

The inventors were next interested more specifically in the effects of various substitutions that the mutants had on their interaction potential with the HLA II molecules. These effects are presented globally for CDRH2 and CDRH3 in FIG. 11A. For each of the two regions, a score unit is counted for each nonhuman core predicted below the 20% threshold for an allele from the panel ([Table 1]). For the CDRH2, the prediction gives a score reduced by more than five times for the mutants 1 and 7 which share the same sequence on this region; for the mutant 2, the effect is even stronger with no cores predicted under the 20% threshold. For CDRH3, a reduction of the predicted scores for the three mutants is also observed; this reduction is over 50% for mutants 1 and 7. The effect of the mutations according to netMHCllpan seems finally greater for CDRH2 than CDRH3. This observation is however more nuanced if one is interested in the effect of these substitutions more specifically on the targeted interaction cores. (FIG. 11B and FIG. 12) For CDRH2 the core “ITWNSGHID” (SEQ ID NO: 12) overlapping the two T cell epitopes identified by Meunier et al. (op. cit.) probably constitutes the modality of interaction with the HLA II molecules. For this core, the results of the prediction are substantially the same for the prediction over the entire region is CDRH2. This therefore serves to confirm that the observed effect is principally carried by the targeted core. (FIG. 11B) For the CDRH3, the cores potentially responsible for the interaction of the identified T cell epitopes are multiple with however three cores mostly predicted for a large number of alleles. (FIG. 11B) A reduction of the number of alleles responding to a 20% threshold for these three cores is observed; this reduction is however greater for the cores B and C. For the core A, the prediction by netMHCIIPan also gives a reduction of the number of affected alleles however it is less marked than for the two other cores.

### CONCLUSION

With this work it was possible to more precisely understand the role of each of the residues from two zones in which the epitopes of adalimumab (CDRH2 and CDRH3) were identified. The YSD platform with Fab format implemented with the high throughput sequencing allowed the functional study of the immunogenic regions by DMS in a first step. Through this first step the inventors were able to observe that, as they had imagined, the strict reduction of immunogenicity by elimination of T cell epitopes does not tend towards functional solutions. The netMHCllpan algorithm mostly proposes substituting hydrophobic residues with small amino acids and/or hydrophilic amino acids. There are however many hydrophobic residues in the CDR and they are particularly important for the structure thereof on which the functionality of the antibody depends directly. It therefore seems difficult to imagine that the substitution of all the hydrophobic amino acids from the CDR can allow retention of the functionality. Having made this observation, the DMS turned out to be even more important for the identification of the functional substitutions. The libraries thus generated and screened based on the DMS data and predictions from the netMHCllpan algorithm made it possible to identify mutants having an increased affinity for TNF alpha and a potentially reduced immunogenicity. The problem of preservation of the functionality during suppression of T cell epitopes located on major regions for interaction with the target can be addressed by getting these mutants. In that way they show that the proposed de-immunization strategy served to reconcile the two non-convergent objectives which are the functionality of the antibody and the reduction of the immunogenicity thereof.

The mutants have a reduction of interaction with the HLA II molecules predicted according to the netMHCllpan algorithm. Because of this, they are less susceptible to being presented by HLA II molecules and recognized by the T cell lymphocytes compared to adalimumab. These mutants thus constitute variants of adalimumab with reduced immunogenic potential and allow overcoming immunogenicity problems encountered with the anti-TNF alpha. These variants could represent a clinical improvement by allowing reduction of the patient immunization rate. Additionally, the inventors were able to show that they have an increased affinity for TNF alpha. To the extent where the biological activity of the anti-TNF antibody—specifically the neutralization of TNF alpha—depends on the affinity thereof for TNF, it can be expected that the mutants from the invention will have a biological activity greater than that of adalimumab. The mutants obtained at the outcome of this work could be considered as potential medication candidates positioning them as an improved version of adalimumab.

1. A variant of a therapeutic anti-TNF alpha antibody comprising variable domains VH and VL of sequences SEQ ID NO: 1 and SEQ ID NO: 2, said variant comprising at least two amino acid substitutions in at least one sequence overlapping one of the CDRH2 or CDRH3 regions determining the complementarity of said VH variable domain;
where said at least two amino acid substitutions in the sequence overlapping the CDRH2 region are selected from the group consisting of:
the substitution of S49 by another amino acid selected from A or G;
the substitution of A50 by another amino acid selected from G, S, T or D;
the substitution of T52 by another amino acid selected from A, N or S;
the substitution S54G; and
the substitution of I57 by another amino acid selected from A, H, N, Q, R, S, T or W;
and when the variant comprises the residue A49 then it also comprises the residue N52, S52 or S52 and the residue G54;
wherein said at least two amino acid substitutions in the sequence overlapping the CDRH3 region are selected from the group consisting of:
the substitution of V89 by L;
the substitution of V95 by another amino acid selected from A, S or T;
the substitution of S96 by another amino acid selected from A, G, H, K, N, Q, R or T;
the substitution of Y97 by H;
the substitution of L98 by T;
the substitution of S99 by P; and
the substitution of T100 by another amino acid selected from P or S;
with the exclusion of variants comprising the residues V89 or L89, V95, K96, Y97, L98, P99 and S100;
V89, V95, A96, Y97, L98, P99 and S100; wherein the positions of said amino acid residues are indicated with reference to the Kabat numbering; and said variant presenting a reduced immunogenic potential and a TNF alpha binding affinity at least equal or superior, compared to the therapeutic anti-TNF alpha antibody from which it is derived.

2. The variant according to claim 1, comprising a combination of substitutions in the sequence overlapping the CDRH2 region selected from:
S54G and I57R;
T52N or T52S, S54G and I57T, I57R, I57Q or I57H;
S49G, T52N and I57H;
S49A or S49G, S54G and I57T or I57R;
S49G, T52N, T52S or T52A; S54G; and I57T, I57R, I57H, I57S, I57Q or I57N; and possibly A50T, A50G or A50S;
S49A, T52N or T52S; S54G; and I57T, I57R, I57H, I57Q, I57S or I57A; and
S49G, A50G, S54G and I57R.

3. The variant according to claim 2, comprising a combination of substitutions in the sequence overlapping the CDRH2 region selected from:
(i) S49G, T52N and I57H; S49A, S54G and I57T; S49G, S54G and I57R; T52N, S54G and I57T;
(ii) S49G, T52N, S54G and I57R; S49G, T52N, S54G and I57H; S49G, T52N, S54G and I57T; S49G, T52N, S54G and I57S; S49G, A50G, T52N and I57H; S49G, T52S, S54G and I57R; S49A, T52N, S54G and I57T; S49G, T52S, S54G and I57N; S49G, T52S, S54G and I57Q; S49G, A50G, S54G and I57R; S49G, T52S, S54G and I57H; S49G, T52S, S54G and I57T; S49G, T52S, S54G and I57S; S49A, T52N, S54G and I57H; and
(iii) S49G, A50T, T52N, S54G and I57S; S49G, A50G, T52N, S54G and I57R; S49G, A50S, T52N, S54G and I57R; S49G, A50D, T52S, S54G and I57T; S49G, A50G, T52S, S54G and I57R; S49G, A50S, T52A, S54G and I57H; S49G, A50S, T52S, S54G and I57R; S49G, A50S, T52A, S54G and I57T; and S49G, A50S, T52A, S54G and I57S.

4. The variant according to claim 1, comprising a combination of substitutions in the sequence overlapping the CDRH3 region selected from:
V95S, V95T or V95A; and
S96T, S96Q, S96N or S96H; and
S99P; and possibly V89L.

5. The variant according to claim 1, comprising a combination of substitutions in the sequence overlapping the CDRH3 region selected from:
(a) S96K and S99P;
(b) V95T, S96T and S99P; V95T, S96K and S99P; V95T, S96R and S99P; V95T, S99P and T100S; V89L, S96K and S99P; S96T, S99P and T100S; S96K, S99P; V95A, S96K and S99P; V95S, S96K and S99P; V95T, S96G and S99P; S96K, S99P and T100P; V95T, S96H and S99P; V95T, S96N and S99P; V95S, S96Q and S99P; V95A, S96H and S99P;
(c) V89L, V95T, S96N and S99P; V95T, S96T, S99P and T100S; V95A, S96H, Y97H and S99P; V89L, S96K, L98T, S99P; S96T, L98T, S99P and T100S; V89L, V95T, S96K and S99P; V95T, S96K, S99P and T100S; V95T, S96R L98T and S99P; V89L, V95T, S96R and S99P; V89L; V95T, S96T and S99P;
(d) V89L, V95T, S96T, S99P and T100S; V89L, S96K, L98T and S99P; V89L, V95T, S96K, S99P and T100S; V89L, V95T, S96R, S99P and T100S.

6. The variant according to claim 5, comprising a combination of substitutions in the sequence overlapping the CDRH3 region selected from: V95T, S96T and S99P; V95T, S96N and S99P; V95S, S96Q and S99P; V95A, S96H and S99P; V95T, S96G and S99P; S96T, L98T, S99P and T100S; V95T, S96R, L98T and S99P; S96K, S99P and T100P; V89L, V95T, S96T and S99P.

7. The variant according to claim 1, comprising at least three substitutions in one of the sequences overlapping the CDRH2 or CDRH3 region.

8. The variant according to claim 7, comprising one of the following combinations of substitutions in the sequences overlapping the CDRH2 and CDRH3 regions:
S49G, T52N, S54G, I57R, V95S, S96Q and S99P;
S49G, A50T, T52N, S54G, I57S, V95T, S96T and S99P;
S49G, T52N, S54G, I57H, V95T, S96T and S99P;
S49G, T52N and I57H, V95T, S96T and S99P;
S49G, A50D, T52S, S54G and I57T, V95T, S96T and S99P;
S49G, T52N, S54G, I57R, V89L, V95T, S96T and S99P;
S49G, T52N, S54G, I57R, V95T, S96N and S99P;
S49G, T52N, S54G, I57R, V95A, S96H and S99P.

9. The variant according to claim 1 further comprising the substitution R90K in the region CDRL3 determining the complementarity of the variable domain VL.

10. The variant according to claim 1 comprising a human IgG heavy chain and a human Kappa light chain.

11. The variant according to claim 1 derived from adalimumab.

12. The variant according to claim 11, comprising a light chain of sequence SEQ ID NO: 2 or 32 and a heavy chain of sequence SEQ ID NO: 24 to 31.

13. An expression vector comprising a polynucleotide coding for a variant according to claim 1.

14. A pharmaceutical composition comprising at least one variant according to claim 1 and a pharmaceutically acceptable vehicle and/or a carrier substance.

15. (canceled)

16. A method for treating an inflammatory or autoimmune disease in a human individual in need thereof, comprising administering to the individual a therapeutically effective amount of the composition according to claim 14.

17. A pharmaceutical composition comprising a vector according to claim 13 and a pharmaceutically acceptable vehicle and/or a carrier substance.

18. A method for treating an inflammatory or autoimmune disease in a human individual in need thereof, comprising administering to the individual a therapeutically effective amount of the composition according to claim 17.


SEQUENCE LISTING (33 sequences)

SEQ ID NO: 1 - protein, 121 aa, artificial sequence [sequence omitted]

SEQ ID NO: 2 - protein, 107 aa, artificial sequence [sequence omitted]

SEQ ID NO: 3 - protein, 451 aa, artificial sequence
  synthetic polypeptide (Adalimumab heavy chain)
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, artificial sequence
  synthetic polypeptide (Adalimumab Light chain)
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 29 aa, artificial sequence
  synthetic peptide (Adalimumab CDRH2 sequence 41-67)
EPGKGLEWVSAITWNSGHIDYADSVEGRF

SEQ ID NO: 6 - protein, 28 aa, artificial sequence
  synthetic peptide (Adalimumab CDRH3 sequence 82A-103)
NSLRAEDTAVYYCAKVSYLSTASSLDYW

SEQ ID NO: 7 - protein, 28 aa, artificial sequence
  synthetic peptide (Adalimumab CDRL3 sequence 73-100)
LTISSLQPEDVATYYCQRYNRAPYTFGQ

SEQ ID NO: 8 - protein, 20 aa, artificial sequence
  synthetic peptide (Adalimumab CDRH2 sequence 46-64)
EWVSAITWNSGHIDYADSVE

SEQ ID NO: 9 - protein, 20 aa, artificial sequence
  synthetic peptide (Adalimumab CDRH3 sequence 89-104)
VYYCAKVSYLSTASSLDYWG

SEQ ID NO: 10 - protein, 16 aa, artificial sequence
  synthetic peptide (Adalimumab CDRH2 sequence 46-60)
EWVSAITWNSGHIDYA

SEQ ID NO: 11 - protein, 18 aa, artificial sequence
  synthetic peptide (Adalimumab CDRH3 sequence 86-100C)
DTAVYYCAKVSYLSTASS

SEQ ID NO: 12 - protein, 9 aa, artificial sequence
  synthetic peptide (Adalimumab CDRH2 T-epitope)
ITWNSGHID

SEQ ID NO: 13 - protein, 9 aa, artificial sequence
  synthetic pepide (Adalimumab mutant 1 and 7 CDRH2 T-epitope)
INWNGGHRD

SEQ ID NO: 14 - protein, 9 aa, artificial sequence
  synthetic peptide (Adalimumab mutant 2 CDRH2 T-epitope)
INWNGGHSD

SEQ ID NO: 15 - protein, 9 aa, artificial sequence
  synthetic peptide (Adalimumab CDRH3 T-epitope (core A))
YYCAKVSYL

SEQ ID NO: 16 - protein, 9 aa, artificial sequence
  synthetic peptide (Adalimumab CDRH3 T-epitope (core B))
VSYLSTASS

SEQ ID NO: 17 - protein, 9 aa, artificial sequence
  synthetic peptide (Adalimumab CDRH3 T-epitope (core C))
YLSTASSLD

SEQ ID NO: 18 - protein, 9 aa, artificial sequence
  synthetic peptide (Adalimumab Mutant 1 CDRH3 T-epitope (core A))
YYCAKSQYL

SEQ ID NO: 19 - protein, 9 aa, artificial sequence
  synthetic peptide (Adalimumab Mutant 2 CDRH3 T-epitope (Core A))
YYCAKTTYL

SEQ ID NO: 20 - protein, 9 aa, artificial sequence
  synthetic peptide (Adalimumab Mutant 7 CDRH3 T-epitope (Core A))
YYCAKAHYL

SEQ ID NO: 21 - protein, 9 aa, artificial sequence
  synthetic peptide (Adalimumab Mutant 1 CDRH3 T-epitope (core B))
SQYLSTASS

SEQ ID NO: 22 - protein, 9 aa, artificial sequence
  synthetic peptide (Adalimumab Mutant 2 CDRH3 T-epitope (core B))
TTYLSTASS

SEQ ID NO: 23 - protein, 9 aa, artificial sequence
  synthetic peptide (Adalimumab Mutant 7 CDRH3 T-epitope (core B))
AHYLSTASS

SEQ ID NO: 24 - protein, 121 aa, artificial sequence
  synthetic polypeptide (Adalimumab Mutant 1 VH)
EVQLVESGGGLVQPGRSLRLSCAASGFTFDDYAMHWVRQAPGKGLEWVGAINWNGGHRDYADSVEGRFTISRDNAKNSLYLQMNSLRAEDTAVYYCAKSQYLPTASSLDYWGQGTLVTVSS

SEQ ID NO: 25 - protein, 121 aa, artificial sequence
  synthetic polypeptide (Adalimumab Mutant 2 VH)
EVQLVESGGGLVQPGRSLRLSCAASGFTFDDYAMHWVRQAPGKGLEWVGTINWNGGHSDYADSVEGRFTISRDNAKNSLYLQMNSLRAEDTAVYYCAKTTYLPTASSLDYWGQGTLVTVSS

SEQ ID NO: 26 - protein, 121 aa, artificial sequence
  synthetic polypeptide (Adalimumab Mutant 3 VH)
EVQLVESGGGLVQPGRSLRLSCAASGFTFDDYAMHWVRQAPGKGLEWVGAINWNGGHHDYADSVEGRFTISRDNAKNSLYLQMNSLRAEDTAVYYCAKTTYLPTASSLDYWGQGTLVTVSS

SEQ ID NO: 27 - protein, 121 aa, artificial sequence
  synthetic polypeptide (Adalimumab Mutant 4 VH)
EVQLVESGGGLVQPGRSLRLSCAASGFTFDDYAMHWVRQAPGKGLEWVGAINWNSGHHDYADSVEGRFTISRDNAKNSLYLQMNSLRAEDTAVYYCAKTTYLPTASSLDYWGQGTLVTVSS

SEQ ID NO: 28 - protein, 121 aa, artificial sequence
  synthetic polypeptide (Adalimumab Mutant 5 VH)
EVQLVESGGGLVQPGRSLRLSCAASGFTFDDYAMHWVRQAPGKGLEWVGDISWNGGHTDYADSVEGRFTISRDNAKNSLYLQMNSLRAEDTAVYYCAKTTYLPTASSLDYWGQGTLVTVSS

SEQ ID NO: 29 - protein, 121 aa, artificial sequence
  synthetic polypeptide (Adalimumab Mutant 6 VH)
EVQLVESGGGLVQPGRSLRLSCAASGFTFDDYAMHWVRQAPGKGLEWVGAINWNGGHRDYADSVEGRFTISRDNAKNSLYLQMNSLRAEDTAVYYCAKTNYLPTASSLDYWGQGTLVTVSS

SEQ ID NO: 30 - protein, 121 aa, artificial sequence
  synthetic polypeptide (Adalimumab Mutant 7 VH)
EVQLVESGGGLVQPGRSLRLSCAASGFTFDDYAMHWVRQAPGKGLEWVGAINWNGGHRDYADSVEGRFTISRDNAKNSLYLQMNSLRAEDTAVYYCAKAHYLPTASSLDYWGQGTLVTVSS

SEQ ID NO: 31 - protein, 121 aa, artificial sequence
  synthetic polypeptide (Adalimumab Mutant 8 VH)
EVQLVESGGGLVQPGRSLRLSCAASGFTFDDYAMHWVRQAPGKGLEWVGAINWNGGHRDYADSVEGRFTISRDNAKNSLYLQMNSLRAEDTALYYCAKTTYLPTASSLDYWGQGTLVTVSS

SEQ ID NO: 32 - protein, 107 aa, artificial sequence
  synthetic polypeptide (Adalimumab variant VL-R90K)
DIQMTQSPSSLSASVGDRVTITCRASQGIRNYLAWYQQKPGKAPKLLIYAASTLQSGVPSRFSGSGSGTDFTLTISSLQPEDVATYYCQKYNRAPYTFGQGTKVEIK

SEQ ID NO: 33 - protein, 9 aa, artificial sequence
  synthetic peptide (Adalimumab Mutant 1, 2 and 7 CDRH3 T-epitope (Core C))
YLPTASSLD